(19) United States
(12) Patent Application Publication
(10) Pub. No.: US 2022/0249388 A1
(43) Pub. Date: Aug. 11, 2022

(54) NANOPARTICLES COMPRISING ENZALUTAMIDE

(71) Applicant: HELM AG, Hamburg (DE)

(72) Inventors: Heiko BRUNNER, Quickborn (DE); Frank GINDULLIS, Klein Offenseth-Sparrieshoop (DE)

(73) Assignee: HELM AG, Hamburg (DE)

(21) Appl. No.: 17/612,863

(22) PCT Filed: May 22, 2020
(86) PCT No.: PCT/EP2020/064268

§ 371 Date: Nov. 19, 2021

(30) Foreign Application Priority Data
May 23, 2019 (EP) 19176304.4
Nov. 14, 2019 (EP) 19209182.5

(51) Int. Cl. A61K 9/51; A61K 31/4166; A61P 35/00

(52) U.S. Cl. CPC A61K 9/51; A61K 31/4166; A61K 9/5123; A61P 35/00; A61K 9/5192; A61K 9/5161; A61K 9/5146

(57) ABSTRACT

The invention relates to nanoparticles comprising Enzalutamide, processes for the preparation of such nanoparticles, pharmaceutical compositions and pharmaceutical dosage forms comprising such nanoparticles, processes for the preparation of such pharmaceutical dosage forms, and uses of the pharmaceutical dosage forms for medical purposes.

The present application claims priority to European patent application no. 19 176 304.4, filed on May 23, 2019, and to European patent application no. 19 209 182.5, filed on Nov. 14, 2019.

The invention relates to nanoparticles comprising Enzalutamide, processes for the preparation of such nanoparticles, pharmaceutical compositions and pharmaceutical dosage forms comprising such nanoparticles, processes for the preparation of such pharmaceutical dosage forms, and uses of the pharmaceutical dosage forms for medical purposes.

Enzalutamide is an androgen receptor signaling inhibitor used as an agent for treating castration-resistant prostate cancer (U.S. Pat. No. 7,709,517). Enzalutamide is provided commercially as soft capsules and tablets (brand name “XTANDI®”). The soft gel capsules are filled with a liquid comprising 40 mg of Enzalutamide per one capsule and pharmaceutical excipients. Tablets comprising 40 or 80 mg Enzalutamide per one tablet and pharmaceutical excipients. The daily dosage is 160 mg, and a patient therefore needs to take four capsules or four 40 mg tablets or two 80 mg tablets daily. A suitable single tablet of reasonable size comprising the prescribed amount of Enzalutamide and having suitable and advantageous solubility and/or dissolution stability and absorption would be advantageous as a suitable alternative to soft capsules.

US 2002/031547 relates to a pharmaceutical composition useful for rapid disintegration, which comprises a sparingly soluble medicament held on a gel-forming water-soluble polymer as a solid dispersion, wherein it contains a salt substance that comprises an alkali and a weak or strong acid and has an endothermic standard enthalpy of solution or heat of solution. Since rapid disintegration of the pharmaceutical composition of the present invention and rapid dissolution of the medicament contained in the preparation can be made in the digestive tracts pH-independently, good bioavailability can be attained.

US 2002/009494 suggests spray dried solid dispersions comprising a sparingly soluble drug and hydroxypropylmethylcellulose acetate succinate (HPMCAS) to provide increased aqueous solubility ands or bioavailability in a use environment.

WO 2014/043208 provides formulations of Enzalutamide and their use for treating hyperproliferative disorders.

Methods of producing microparticles and nanoparticles are described in various patent applications and patents, for example in U.S. Pat. Nos. 5,833,891, 5,534,270, 6,862,890, 6,177,103, DE 10 2005 053 862, U.S. Pat. Nos. 5,833,891, 5,534,270, 6,862,890, 6,177,103, DE 10 2005 017 777 and DE 10 2005 053 862.

V. Wilson et al., Journal of Controlled Release, 292 (2018) 172-182 relates to amorphous solid dispersions of Enzalutamide that are prepared with hydrophilic polymers hydroxypropylmethylcellulose acetate succinate and copovidone (PVP/VA). The formulations were tested in vivo in rats using oral dosing of amorphous solid dispersions suspensions. Amorphous solid dispersions that underwent crystallization showed lower plasma exposures. Differences were also observed between amorphous solid dispersions that dissolved to form nanosized amorphous drug aggregates versus those that dissolved to yield only supersaturated solutions, with the former outperforming the latter in terms of the plasma exposure. The authors conclude that these observations highlight the importance of thoroughly understanding the phase behavior of an amorphous formulation following dissolution and the need to discriminate between different types of precipitation, specifically crystallization versus glass liquid phase separation to form nanosized amorphous aggregates.

Ch. Thangavel et al., Mol. Pharm. 2018, 15(5), 1778-1790 relates to anti-PSMA-conjugated hybrid antiandrogen nanoparticles and their therapeutic efficacy and cellular toxicity.

WO 02/60275 A1 describes methods of producing nanoparticles in which two immiscible liquids are charged electrically so as to achieve encapsulation. In this case, the use of toxic substances is not ruled out, meaning that product quality may suffer considerably as a result. Particle size, moreover, cannot be controlled with this method.

US 2009/0214655 A1 also describes the use of two immiscible liquids, Although a microreactor is used there to produce the nanoparticles, only the production of emulsions is described. In addition, the nanoparticles are produced in a liquid-filled space in which, once again, it is impossible to control either particle size or the particle properties. Furthermore, the device can easily become blocked due to the fact that the reactions are carried out in microchannels.

The properties of the known formulations of Enzalutamide are not satisfactory in every respect and there is a demand for pharmaceutical compositions and dosage forms that contain Enzalutamide and that are advantageous over the prior art, e.g. with respect to the release profile and/or drug load.

It is therefore an object of the invention to provide pharmaceutical compositions and dosage forms that contain Enzalutamide and that are advantageous over the prior art. In one aspect, the present invention aims at providing pharmaceutical compositions and dosage forms providing immediate release of Enzalutamide. In another aspect, the present invention aims at providing pharmaceutical compositions and oral dosage forms having a comparatively high drug load, preferably up to about 160 mg Enzalutamide per dosage form. In still another aspect, the present invention aims at providing pharmaceutical compositions and oral dosage forms that contain Enzalutamide and that show high bioavailability, preferably upon oral administration. In yet another aspect, the present invention aims at providing pharmaceutical compositions and oral dosage forms that can be easily manufactured and are stable.

This object has been achieved by the subject-matter of the patent claims.

It has been surprisingly found that nanoparticles comprising Enzalutamide can be prepared by precipitation from solvents (e.g. acetone, THF) when admixed with suitable non-solvents (e.g. water). Further, it has been surprisingly found that the size of the thus obtained nanoparticles can be influenced by selecting proper excipients which also stabilize the nanoparticles. Still further, it has been surprisingly found that depending upon size, concentration and excipients, nanoparticles comprising Enzalutamide can be prepared that completely or nearly completely disperse from suspension into fasted state simulating fluid (FaSSIF) thereby indicating that such nanoparticles will likely provide good bioavailability of Enzalutamide when administered in vivo.

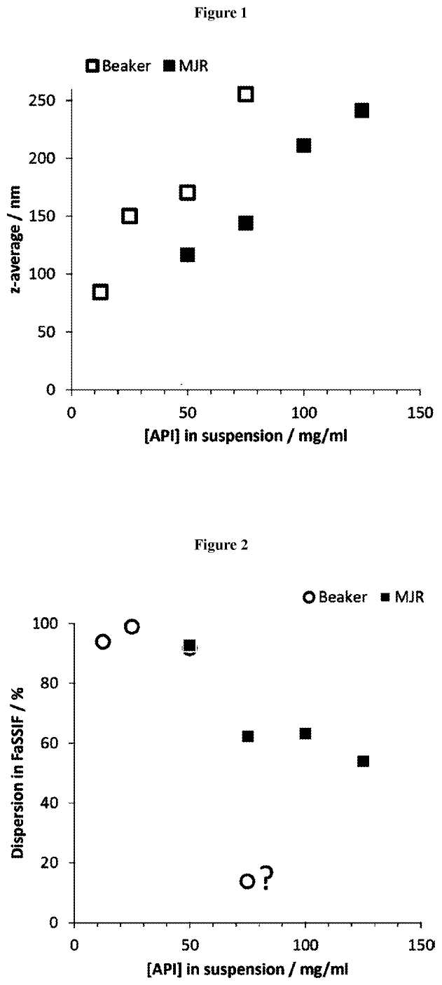

FIG. 1 shows the z-average particle size in dependence of the concentration of Enzalutamide (API) in suspension for nanoparticles prepared from the system Pluronic® F127/Soluplus®/THF by precipitation in a beaker and microjet reactor technology, respectively.

FIG. 2 shows the percentage of dispersion in FaSSIF in dependence of the concentration of Enzalutamide (API) in suspension for nanoparticles prepared from the system Pluronic® F127/Soluplus®/THF by precipitation in a beaker and microjet reactor technology, respectively.

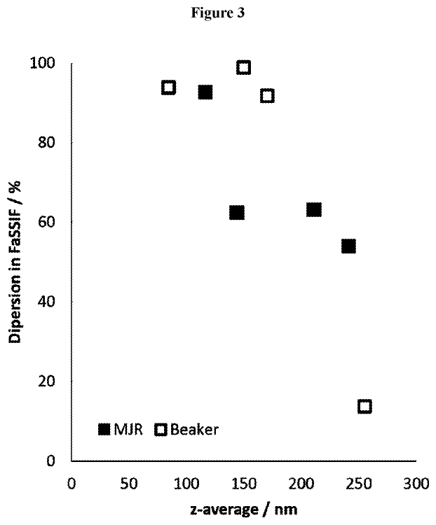

FIG. 3 shows the percentage of dispersion in FaSSIF in dependence of the z-average particle size for nanoparticles prepared from the system Pluronic® F127/Soluplus®/THF by precipitation in a beaker and microjet reactor technology, respectively.

A first aspect of the invention relates to nanoparticles comprising Enzalutamide.

The nanoparticles according to the invention comprise Enzalutamide. Enzalutamide is a nonsteroidal antiandrogen (NSAA) medication which is used in the treatment of prostate cancer. It is indicated for use in conjunction with castration in the treatment of metastatic castration-resistant prostate cancer (mCRPC) and nonmetastatic castration-resistant prostate cancer. Enzalutamide is an antiandrogen, and acts as an antagonist of the androgen receptor. It prevents the effects of androgens in the prostate gland.

Enzalutamide (CAS 915087-33-1) has the following chemical structure:

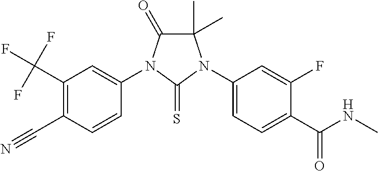 [SMILES: CNC(=O)c1ccc(N2C(=S)N(c3ccc(C#N)c(C(F)(F)F)c3)C(=O)C2(C)C)cc1F]

Enzalutamide is a white-to-off white solid that is insoluble in water. One crystalline form and four solvates have been observed so far. For the purpose of the specification, unless expressly stated otherwise, the term “Enzalutamide” refers to Enzalutamide, its non-salt form, physiologically acceptable salts, co-crystals, polymorphs and/or solvates thereof.

Preferably, the nanoparticles according to the invention contain Enzalutamide in its non-salt form.

Unless expressly stated otherwise, all dosages and weight percent used herein are based upon the equivalent weight relative to the non-salt form and non-solvate form and non co-crystal form of Enzalutamide, i.e. the additional weight of the salt moiety or solvent moiety or co-crystal moiety is not taken into account for the quantification.

Preferably, the nanoparticles according to the invention are solid.

Preferably, the Enzalutamide within the nanoparticles has a degree of crystallinity of at least 10%, preferably at least 20%, more preferably at least 30%. Preferably, the Enzalutamide within the nanoparticles has a degree of crystallinity of at least 40%, preferably at least 50%, more preferably at least 60%. Preferably, the Enzalutamide within the nanoparticles has a degree of crystallinity of at least 70%, preferably at least 80%, more preferably at least 90%, still more preferably at least 95%, yet more preferably at least 99% and in particular about 100%.

Methods for determining the degree of crystallinity are known to a skilled person and involve e.g. x-ray powder diffraction analysis or differential scanning calorimetry (DSC).

In another preferred embodiment, the Enzalutamide within the nanoparticles is substantially non-crystalline, i.e. amorphous. According to this embodiment, the Enzalutamide within the nanoparticles preferably has a degree of crystallinity of at most 20%, preferably at most 15%, more preferably at most 10%. Preferably, the Enzalutamide within the nanoparticles has a degree of crystallinity of at most 5.0%, preferably at most 2.5%, more preferably at most 1.0%.

While it is principally contemplated that the nanoparticles according to the invention may contain other pharmacologically active ingredients besides the Enzalutamide, Enzalutamide is preferably the sole pharmacologically active ingredient that is contained in the nanoparticles. In this context, pharmacologically active ingredients are other substances that are useful in treating the same or related disorders and diseases and conditions as Enzalutamide. Thus, compounds having a physiological but no pharmacological effect such as sodium chloride, vitamins and the like, are not to be regarded as pharmacologically active ingredients in the above meaning.

Preferably, the nanoparticles according to the invention and the Enzalutamide contained therein are not conjugated to antigens, e.g. for the purposes of drug targeting. In particular, the nanoparticles according to the invention are not encapsulated in a prostate specific membrane antigen (PSMA), i.e. are not coated with PSMA.

The particle size of the nanoparticles according to the invention is not particularly limited. However, the term “nanoparticles” already implies a certain particle size in the nanometer scale. When the nanoparticles have a core shell structure, the particle size is determined by core and shell together. The term “nanoparticles” typically means particles having a diameter comprised between 1 and 1000 nm in size. Said diameter can be determined according to methods known to the skilled person in the art, for example with Dynamic Light Scattering (DLS), and Transmission Electron Microscopy (TEM). Advantageously, the nanoparticles according to the invention have a diameter comprised between 20 and 1000 nm, more advantageously between 30 and 500 nm, even more advantageously between 40 and 350 nm, preferably between 60 and 250 nm.

Preferably, the nanoparticles according to the invention have a z-average particle size Dz determined in accordance with ISO 22412:2008 Particle Size Analysis—Dynamic Light Scattering of not more than 1000 nm, preferably not more than 900 nm, more preferably not more than 800 nm. Preferably, the nanoparticles according to the invention have a z-average particle size Dz of not more than 700 nm, preferably not more than 600 nm, more preferably not more than 500 nm. Preferably, the nanoparticles according to the invention have a z-average particle size Dz of not more than 400 nm, preferably not more than 300 nm, more preferably not more than 200 nm. Preferably, the nanoparticles according to the invention have a z-average particle size Dz of not more than 150 nm, preferably not more than 125 nm, more preferably not more than 100 nm.

In preferred embodiments, the nanoparticles according to the invention have a z-average particle size Dz within the range of 60±50 nm, or 70±50 nm, or 80±50 nm, or 90±50 nm, or 100±50 nm, or 110±50 nm, or 120±50 nm, or 130±50 nm, or 140±50 nm. In preferred embodiments, the nanoparticles according to the invention have a z-average particle size Dz within the range of 60±30 nm, or 70±30 nm, or 80±30 nm, or 90±30 nm, or 100±30 nm, or 110±30 nm, or 120±30 nm, or 130±30 nm, or 140±30 nm. In preferred embodiments, the nanoparticles according to the invention have a z-average particle size Dz within the range of 60±10 nm, or 70±10 nm, or 80±10 nm, or 90±10 nm, or 100±10 nm, or 110±10 nm, or 120±10 nm, or 130±10 nm, or 140±10 nm.

In other preferred embodiments, the nanoparticles according to the invention have a z-average particle size Dz within the range of 200±150 nm, or 200±100 nm, or 200±50 nm; or 300±150 nm, or 300±100 nm, or 300±50 nm; or 400±150 nm, or 400±100 nm, or 400±50 nm; or 500±150 nm, or 500±100 nm, or 500±50 nm; or 600±150 nm, or 600±100 nm, or 600±50 nm; or 700±150 nm, or 700±100 nm, or 700±50 nm; or 800±150 nm, or 800±100 nm, or 800±50 nm; or 900±150 nm, or 900±100 nm, or 900±50 nm.

In a particularly preferred embodiment, the nanoparticles according to the invention have a z-average particle size Dz within the range of 850±150 nm, or 850±100 nm, or 850±50 nm.

The z-average particle size Dz is the intensity based harmonic: mean (23) and methods for determining Dz are known to the skilled person such as laser scattering. According to the invention, Dz is preferably determined in accordance with ISO 22412:2008 Particle Size Analysis—Dynamic Light Scattering.

The width of the particle size distribution in suspension is characterized by the “polydispersity” or “PDI” of the nanoparticles, which is defined as the relative variance in the correlation decay rate distribution, as is known by one skilled in the art. The polydispersity index (PDI) can also be calculated from the cumulants analysis of the DLS measured intensity autocorrelation function as defined in IS022412:2008. Preferably, the polydispersity of the nanoparticles according to the invention is less than 0.6, or less than 0.5, or less than 0.4, or less than 0.3, or less than 0.2, or less than 0.1.

Nanoparticles essentially only consisting of Enzalutamide are typically not stable. Thus, the nanoparticles according to the invention preferably additionally comprise one or more pharmaceutical excipients independently of one another selected from the group consisting of surfactants and polymers.

It is contemplated that the nanoparticles according to the invention can exist in a number of different configurations.

In one embodiment, the nanoparticles according to the invention comprise a core, the core comprising Enzalutamide or a pharmaceutically acceptable salt thereof. As used herein, the term “core” refers to the interior portion of the nanoparticle. The nanoparticles according to this embodiment also have a “surface” or an “outer” portion. The nanoparticles can, thus, have a core (i.e., the interior portion) and a surface or outer portion substantially surrounding the core. In one embodiment of the invention, the core contains essentially the total amount of Enzalutamide or a pharmaceutically acceptable salt thereof, optionally together with one or more excipients, and the outer portion is substantially comprised of one or more excipients but essentially contains no Enzalutamide or a pharmaceutically acceptable salt thereof

In another embodiment, the concentration of Enzalutamide or a pharmaceutically acceptable salt thereof can vary throughout the nanoparticles with the concentration of Enzalutamide or a pharmaceutically acceptable salt thereof being highest, for example, at the core. For example, the nanoparticles of the invention can comprise a matrix of one or more excipients and Enzalutamide or a pharmaceutically acceptable salt thereof, such that an amount of Enzalutamide or a pharmaceutically acceptable salt thereof can be dispersed in the outer portion of the nanoparticle and an amount of the excipient or combination of excipients can be dispersed within the core of the nanoparticle, or combinations thereof. Thus, in some embodiments, Enzalutamide or a pharmaceutically acceptable salt thereof can be associated with at least part of the excipient outer portion. Some amount of Enzalutamide or a pharmaceutically acceptable salt thereof can, therefore, be associated with the surface of, encapsulated within, surrounded by, and/or dispersed or diffused throughout the excipient outer portion of the nanoparticles.

In some embodiments, materials may be adsorbed to the surface portion of the nanoparticle. Materials adsorbed to the surface portion of the nanoparticle are considered part of the nanoparticle, but are distinguishable from the core of the nanoparticle. Methods to distinguish materials present in the core versus materials adsorbed to the surface portion of the nanoparticle include (1) thermal methods, such as differential scanning calorimetry (DSC); (2) spectroscopic methods, such as X-ray diffraction (XRD), X-ray photoelectron spectroscopy (XPS), transmission electron microscopy (TEM) with energy dispersive X-ray (EDX) analysis, Fourier transform infra red (FTIR) analysis, and Raman spectroscopy; (3) chromatographic techniques, such as high performance liquid chromatography (HPLC), and gel-permeation chromatography (GPC); and (4) other techniques known in the art.

In preferred embodiments, the nanoparticles according to the invention comprise
(i) at least one surfactant and at least one polymer;
(ii) at least two different surfactants; and/or
(iii) at least two different polymers.

The weight content of all pharmaceutical excipients that are contained in the nanoparticles according to the invention is not particularly limited.

Preferably, the total content of all pharmaceutical excipients that are contained in the nanoparticles is not more than 90 wt.-%, preferably not more than 85 wt.-%, more preferably not more than 80 wt.-%, in each case relative to the total weight of the nanoparticles. Preferably, the total content of all pharmaceutical excipients that are contained in the nanoparticles is not more than 75 wt.-%, preferably not more than 70 wt.-%, more preferably not more than 65 wt.-%, in each case relative to the total weight of the nanoparticles. Preferably, the total content of all pharmaceutical excipients that are contained in the nanoparticles is not more than 60 wt.-%, preferably not more than 55 wt.-%, more preferably not more than 50 wt.-%, in each case relative to the total weight of the nanoparticles. Preferably, the total content of all pharmaceutical excipients that are contained in the nanoparticles is not more than 45 wt.-%, preferably not more than 40 wt.-%, more preferably not more than 35 wt.-%, in each case relative to the total weight of the nanoparticles. Preferably, the total content of all pharmaceutical excipients that are contained in the nanoparticles is not more than 30 wt.-%, preferably not more than 25 wt.-%, more preferably not more than 20 wt.-%, in each case relative to the total weight of the nanoparticles.

Preferably, the total content of all pharmaceutical excipients that are contained in the nanoparticles is at least 0.5 wt.-%, preferably at least 1.0 wt.-%, more preferably at least 1.5 wt.-%, in each case relative to the total weight of the nanoparticles. Preferably, the total content of all pharmaceutical excipients that are contained in the nanoparticles is at least 2.5 wt.-%, preferably at least 5.0 wt.-%, more preferably at least 7.5 wt.-%, in each case relative to the total weight of the nanoparticles. Preferably, the total content of all pharmaceutical excipients that are contained in the nanoparticles is at least 10 wt.-%, preferably at least 20 wt.-%, more preferably at least 30 wt.-%, in each case relative to the total weight of the nanoparticles.

In preferred embodiments, the total weight content of all pharmaceutical excipients that are contained in the nanoparticles is within the range of 25±20 wt.-%, or 30±20 wt.-%, or 35±20 wt.-%, or 40±20 wt.-%, or 45±20 wt.-%, or 50±20 wt.-%, or 55±20 wt.-%, or 60±20 wt.-%, or 65±20 wt.-%, or 70±20 wt.-%, in each case relative to the total weight of the nanoparticles. In preferred embodiments, the total weight content of all pharmaceutical excipients that are contained in the nanoparticles is within the range of 25±10 wt.-%, or 30±10 wt.-%, or 35±10 wt.-%, or 40±10 wt.-%, or 45±10 wt.-%, or 50±10 wt.-%, or 55±10 wt.-%, or 60±10 wt.-%, or 65±10 wt.-%, or 70±10 wt.-%, in each case relative to the total weight of the nanoparticles. In preferred embodiments, the total weight content of all pharmaceutical excipients that are contained in the nanoparticles is within the range of 25±5 wt.-%, or 30±5 wt.-%, or 35±5 wt.-%, or 40±5 wt.-%, or 45±5 wt.-%, or 50±5 wt.-%, or 55±5 wt.-%, or 60±5 wt.-%, or 65±5 wt.-%, or 70±5 wt.-%, in each case relative to the total weight of the nanoparticles.

In some embodiments, the nanoparticles are stabilized with one or more water soluble (e.g., hydrophilic) excipients, one or more water insoluble (e.g., lipophilic) excipients or a combination of one or more water soluble and one or more water insoluble excipients. Examples of water soluble excipients include, but are not limited to, vitamin E TPGS, polysorbate 80, polysorbate 20, Triton X-100, lauryl glucoside, NP-40, oleyl alcohol, sorbitans (monosterate tristearate), stearyl alcohol, nonoxynols, Cremophore (RH 60 or EL), Solutol HS 15, plutonic acid, sodium dodecyl sulfate (SDS), bile acid salts, polyethylene glycol and polypropylene glycol and their combinations. Bile acid salts are preferably selected from the group of salts of cholic acid, chenodeoxycholic acid, deoxycholic acid and urodeoxycholic acid. Examples of water insoluble excipients include, but are not limited to, vitamin E, and its derivatives, bile acid and its derivatives and phospholipid derivatives, lecithin, lysolecithin, phosphotidylserine, glycerophosphocholine, oleic acid, glycerol, inositol, diethylenetriaminepentaaceticacid, polyoxyethylene castor, polyoxyethylenehydrogenated castor oil base, polyoxyethylene sorbitan monolaurate and combinations thereof.

Preferably, the nanoparticles according to the invention comprise one or more surfactants.

In a preferred embodiment, the nanoparticles according to the invention comprise a single surfactant. In another preferred embodiment, nanoparticles according to the invention comprise two, three or four different surfactants.

The weight content of all surfactants that are contained in the nanoparticles according to the invention is not particularly limited.

Preferably, the total content of the one or more surfactants that are contained in the nanoparticles is not more than 20 wt.-%, preferably not more than 15 wt.-%, more preferably not more than 10 wt.-%, in each case relative to the total weight of the nanoparticles. Preferably, the total content of the one or more surfactants that are contained in the nanoparticles is not more than 7.5 wt.-%, preferably not more than 5.0 wt.-%, more preferably not more than 2.5 wt.-%, in each case relative to the total weight of the nanoparticles. Preferably, the total content of the one or more surfactants that are contained in the nanoparticles is not more than 1.5 wt.-%, preferably not more than 1.0 wt.-%, more preferably not more than 0.5 wt.-%, in each case relative to the total weight of the nanoparticles.

Preferably, the total content of the one or more surfactants that are contained in the nanoparticles is at least 0.01 wt.-%, preferably at least 0.05 wt.-%, more preferably at least 0.1 wt.-%, in each case relative to the total weight of the nanoparticles. Preferably, the total content of the one or more surfactants that are contained in the nanoparticles is at least 0.2 wt.-%, preferably at least 0.3 wt.-%, more preferably at least 0.4 wt.-%, in each case relative to the total weight of the nanoparticles. Preferably, the total content of the one or more surfactants that are contained in the nanoparticles is at least 0.5 wt.-%, preferably at least 0.6 wt.-%, more preferably at least 0.7 wt.-%, in each case relative to the total weight of the nanoparticles.

In preferred embodiments, the total weight content of all surfactants that are contained in the nanoparticles is within the range of 0.10±0.05 wt.-%, or 0.15±0.05 wt.-%, or 0.20±0.05 wt.-%, or 0.25±0.05 wt.-%, or 0.30±0.05 wt.-%, or 0.35±0.05 wt.-%, or 0.40±0.05 wt.-%, or 0.45±0.05 wt.-%, or 0.50±0.05 wt.-%, or 0.55±0.05 wt.-%, or 0.60±0.05 wt.-%, or 0.65±0.05 wt.-%, or 0.70±0.05 wt.-%, or 0.75±0.05 wt.-%, or 0.80±0.05 wt.-%, or 0.85±0.05 wt.-%, or 0.90±0.05 wt.-%, or 0.95±0.05 wt.-%, in each case relative to the total weight of the nanoparticles.

The properties of surfactants may be described by their hydrophilic-lipophilic-balance (HLB).

Preferably, the one or more surfactants comprise or essentially consist of a surfactant having a HLB value of at least 10, preferably at least 15, more preferably at least 20. Preferably, the one or more surfactants comprise or essentially consist of a surfactant having a HLB value of at least 25, preferably at least 30, more preferably at least 32. Preferably, the one or more surfactants comprise or essentially consist of a surfactant having a HLB value of at least 34, preferably at least 36, more preferably at least 38.

Preferably, the one or more surfactants comprise or essentially consist of a surfactant having a HLB value of not more than 40, preferably not more than 38, more preferably not more than 35. Preferably, the one or more surfactants comprise or essentially consist of a surfactant having a HLB value of not more than 33, preferably not more than 30, more preferably not more than 28. Preferably, the one or more surfactants comprise or essentially consist of a surfactant having a HLB value of not more than 25, preferably not more than 23, more preferably not more than 20.

In preferred embodiments, the one or more surfactants comprise or essentially consist of a surfactant having a HLB value within the range of 12±10, or 14±10, or 16±10, or 18±10, or 20±10, or 22±10, or 24±10, or 26±10, or 28±10, or 30±10. In preferred embodiments, the one or more surfactants comprise or essentially consist of a surfactant having a HLB value within the range of 12±5, or 14±5, or 16±5, or 18±5, or 20±5, or 22±5, or 24±5, or 26±5, or 28±5, or 30±5, or, or 32±5, or 34±5.

The properties of surfactants may also be described by their charge. In a preferred embodiment, the one or more surfactants comprise or essentially consist of a nonionic surfactant. In a preferred embodiment, the one or more surfactants comprise or essentially consist of an anionic surfactant. In a preferred embodiment, the one or more surfactants comprise or essentially consist of a cationic surfactant. In a preferred embodiment, the one or more surfactants comprise or essentially consist of an amphoteric surfactant.

In a particularly preferred embodiment, the one or more surfactants comprise or essentially consist of a nonionic surfactant. Preferably, the nonionic surfactant is selected from the group consisting of
straight or branched chain fatty alcohols; preferably selected from cetyl alcohol, cetostearyl alcohol, stearyl alcohol, oleyl alcohol, octyldodecanol, or 2-hexyldecane-1-ol;
sterols; preferably cholesterol;
lanolin alcohols;
partial fatty acid esters of multivalent alcohols, e.g. glycerol fatty acid monoesters or glycerol fatty acid diesters; preferably selected from glycerol behenate, glycerol dibehenate, glycerol distearate, glycerol monocaprylate, glycerol monolinoleate, glycerol mono oleate, glycerol monostearate, ethylene glycol monopalmitostearate, ethylene glycol stearate, diethylene glycol palmitostearate, diethylene glycol stearate, propylene glycol dicaprylocaprate, propylene glycol dilaurate, propylene glycol monocaprylate, propylene glycol monolaurate, propylene glycol monopalmitostearate, propylene glycol monostearate, pentaerythritol monostearate, superglycerinated fully hydrogenated rapeseed oil;
partial fatty acid esters of sorbitan; preferably selected from sorbitan monolaurate, sorbitan monopalmitate, sorbitan monostearate, sorbitan tristearate, sorbitan monooleate, sorbitan sesquioleate, sorbitan trioleate;
partial fatty acid esters of polyoxyethylene sorbitan, (polyoxyethylene-sorbitan-fatty acid esters), e.g. fatty acid monoesters of polyoxyethylene sorbitan, a fatty acid diesters of polyoxyethylene sorbitan, or a fatty acid triesters of polyoxyethylene sorbitan; such as mono- and tri-lauryl, palmityl, stearyl and oleyl esters; preferably selected from polyoxyethylene(20)sorbitan monolaurate, polyoxyethylene(4)sorbitan monolaurate, polyoxyethylene(20)sorbitan monopalmitate, polyoxyethylene(20)sorbitan monostearate, polyoxyethylene(20)sorbitan tristearate, polyoxyethylene(20)sorbitan monooleate, polyoxyethylene(5)sorbitan monooleate, polyoxyethylene(20)sorbitan trioleate;
polyoxyethyleneglycerole fatty acid esters, e.g. mixtures of mono-, di- and triesters of glycerol and di- and monoesters of macrogols having molecular weights within the range of from 200 to 4000 g/mol; preferably selected from macro2olglyeerolcaprylocaprate, macrogolglycerollaurate, macrogolglycerolococoate, macrogolglycerollinoleate, macrogol-20-glycerolmonostearate, macrogol-6-glycerolcaprylocaprate, macrogoiglycerololeate; macrogolglycerolstearate, macrogolglycerolhydroxystearate, macrogolglycerolrizinoleate;
polyoxyethylene fatty acid esters; preferably selected from macrogololeate, macrogolstearate, macrogol-15-hydroxystearate, polyoxyethylene esters of 12-hydroxvstearic acid;
fatty alcohol ethers of polyoxyethylene; preferably selected from polyoxyethylene lauryl ether, polyoxyethylene cetyl ether, polyoxyethylene stearyl ether, polyoxyethylene oleyl ether, polyoxyethylene cetostearyl ether, lauromacrogol 400, macrogol oleyl ether, macrogol stearyl ether;
reaction products of a natural or hydrogenated castor oil and ethylene oxide such as those commercialized as Cremophor, and
polyoxypropylene-polyoxyethylene blockcopolymers (poloxamers); preferably according to the following general formula

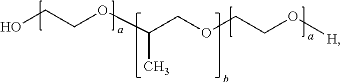

wherein a is an integer independently within the range of from 2 to 130, preferably from 90 to 110; and wherein b is an integer within the range of from 15 to 67, preferably from 46 to 66;
polyglycolyzed glycerides; preferably selected from those commercialized as Gelucire®, Labrasol®;
fatty acid esters of sucrose; preferably selected from sucrose distearate, sucrose dioleate, sucrose dipalmitate, sucrose monostearate, sucrose monopalmitate, sucrose monooleate, sucrose monomyristate, sucrose mololaurate;
fatty acid esters of polyglycerol; preferably selected from polyglycerol oleate polyglycerol dioleate, polyglycerol poly-12-hydroxystearate, triglycerol di-isostearate; and
polyoxyethylene esters of D-α-tocopheryl succinate: preferably D-α-tocopherol polyethylene glycol 1000 succinate.

In a particularly preferred embodiment, the nonionic surfactant is a polyoxyethylene ester of D-α-tocopheryl succinate; preferably D-α-tocopherol polyethylene glycol 1000 succinate. Such a surfactant is commercially available e.g. under the trade name Vitamin E TPGS.

In another particularly preferred embodiment, the nonionic surfactant is a polyoxypropylenepolyoxyethylene blockcopolymers (poloxamers); preferably according to the following general formula

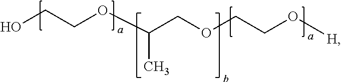

wherein a is an integer independently within the range of from 2 to 130, preferably from 90 to 110, more preferably about 101; and wherein b is an integer within the range of from 15 to 67, preferably from 46 to 66, more preferably about 56; preferably poloxamer 407. Poloxamers are commercially available under the trade names Pluronic®, Kolliphor®, Lutrol®.

In another particularly preferred embodiment, the one or more surfactants comprise or essentially consist of an anionic surfactant. Preferably, the anionic surfactant is selected from the group consisting of
alkyl sulfate salts; preferably selected from sodium lauryl sulfate (sodium dodecyl sulfate), sodium cetyl sulfate, sodium cetyistearyl sulfate, sodium stearyl sulfate, sodium dioctylsulfosuccinate (docusate sodium); and the corresponding potassium or calcium salts thereof:
fatty acid salts; preferably selected from stearic acid salts, oleic acid salts; and
salts of cholic acid; preferably selected from sodium deoxycholate, sodium glycocholate, sodium taurocholate and the corresponding potassium or ammonium salts; particularly preferred is sodium deoxycholate.

In a particularly preferred embodiment, the anionic surfactant is an alkyl sulfate salt; preferably of the general formula CₙH₂ₙ₊₁O—SO₃⁻M⁻, wherein n is an integer of from 8 to 30, preferably 10 to 24, more preferably 12 to 18; and M is selected from Li⁺, Na⁺, NH₄⁺, ½Mg²⁺ and ½ Ca²⁻; preferably sodium dodecyl sulfate. Preferably, the anionic surfactant is sodium dodecyl sulfate.

In another particularly preferred embodiment, the anionic surfactant is a salt of cholic acid; preferably selected from sodium deoxycholate, sodium glycocholate, sodium taurocholate and the corresponding potassium or ammonium salts; particularly preferred is sodium deoxycholate.

Preferably, the nanoparticles according to the invention comprise one or more polymers. These polymers typically have disperse molecular weight distributions.

In a preferred embodiment, the nanoparticles according to the invention comprise a single polymer. In another preferred embodiment, nanoparticles according to the invention comprise two, three or four different polymers.

The weight content of all polymers that are contained in the nanoparticles according to the invention is not particularly limited.

Preferably, the total content of the one or more polymers that are contained in the nanoparticles is not more than 45 wt.-%, preferably not more than 40 wt.-%, more preferably not more than 35 wt.-%, in each case relative to the total weight of the nanoparticles. Preferably, the total content of the one or more polymers that are contained in the nanoparticles is not more than 30 wt.-%, preferably not more than 25 wt.-%, more preferably not more than 20 wt.-%, in each case relative to the total weight of the nanoparticles. Preferably, the total content of the one or more polymers that are contained in the nanoparticles is not more than 15 wt.-%, preferably not more than 10 wt.-%, more preferably not more than 5.0 wt.-%, in each case relative to the total weight of the nanoparticles.

In a particularly preferred embodiment, the nanoparticles contain a graft copolymer of polyethyleneglycol, polyvinyleaprolactant, and polyvinylacetate (e.g. Soluplus®), whereas the total content of said graft polymer that is contained in the nanoparticles is not more than 6.0 wt.-%, preferably not more than 5.5 wt.-%, more preferably not more than 5.0 wt.-%, still more preferably not more than 4.5 wt.-%, yet more preferably not more than 4.0 wt.-%, even more preferably not more than 3.5 wt.-%, most preferably not more than 3.0 wt.-%, and in particular not more than 2.5 wt.-%, in each case relative to the total weight of the nanoparticles.

Preferably, the total content of the one or more polymers that are contained in the nanoparticles is at least 1.0 wt.-%, preferably at least 1.5 wt.-%, or at least 2.0 wt.-%, or at least 2.5 wt.-%, more preferably at least 5.0 wt.-%, in each case relative to the total weight of the nanoparticles. Preferably, the total content of the one or more polymers that are contained in the nanoparticles is at least 7.5 wt.-%, preferably at least 10 wt.-%, more preferably at least 12.5 wt.-%, in each case relative to the total weight of the nanoparticles. Preferably, the total content of the one or more polymers that are contained in the nanoparticles is at least 15 wt.-%, preferably at least 17.5 wt.-%, more preferably at least 20 wt.-%, in each case relative to the total weight of the nanoparticles.

In preferred embodiments, the total content of the one or more polymers that are contained in the nanoparticles is within the range of 2.5±2.0 wt.-%, or 3.0±2.0 wt.-%, or 3.5±2.0 wt.-%, or 4.0±2.0 wt.-%, or 4.5±2.0 wt.-%, or 5.0±2.0 wt.-%, or 5.5±2.0 wt.-%, or 6.0±2.0 wt.-%, or 6.5±2.0 wt.-%, or 70±20 wt.-%, in each case relative to the total weight of the nanoparticles. In preferred embodiments, the total content of the one or more polymers that are contained in the nanoparticles is within the range of 25±20 wt.-%, or 30±20 wt.-%, or 35±20 wt.-%, or 40±20 wt.-%, or 45±20 wt.-%, or 50±20 wt.-%, or 55±20 wt.-%, or 60±20 wt.-%, or 65±20 wt.-%, or 70±20 wt.-%, in each case relative to the total weight of the nanoparticles. In preferred embodiments, the total content of the one or more polymers that are contained in the nanoparticles is within the range of 15±10 wt.-%, or 20±10 wt.-%, 25±10 wt.-%, or 30±10 wt.-%, or 35±10 wt.-%, or 40±10 wt.-%, or 45±10 wt.-%, or 50±10 wt.-%, or 55±10 wt.-%, or 60±10 wt.-%, or 65±10 wt.-%, or 70±10 wt.-%, in each case relative to the total weight of the nanoparticles. In preferred embodiments, the total content of the one or more polymers that are contained in the nanoparticles is within the range of 10±5 wt.-%, or 15±5 wt.-%, or 20±5 wt.-%, or 25±5 wt.-%, or 30±5 wt.-%, or 35±5 wt.-%, or 40±5 wt.-%, or 45±5 wt.-%, or 50±5 wt.-%, or 55±5 wt.-%, or 60±5 wt.-%, or 65±5 wt.-%, or 70±5 wt.-%, in each case relative to the total weight of the nanoparticles.

In preferred embodiments, the one or more polymers comprise or essentially consist of a polymer selected from the group consisting of
neutral non-cellulosic polymers; preferably selected from vinyl polymers and copolymers having substituents of hydroxyl, alkylacyloxy, and cyclic amido polyvinyl alcohols that have at least a portion of their repeat units in the unhydrolyzed (vinyl acetate) form; polyvinyl alcohol polyvinyl acetate copolymers; polyvinyl pyrrolidone; polyvinylpyrrolidone vinyl acetate; and polyethylene polyvinyl alcohol copolymers;
ionizable non-cellulosic polymers; preferably carboxylic acid-functionalized vinyl polymers; preferably selected from carboxylic acid functionalized polymethacrylates and carboxylic acid functionalized polyacrylates; amine-functionalized polyacrylates and polymethacrylates; proteins; and carboxylic acid functionalized starches;
amphiphilic non-cellulosic polymers; preferably selected from acrylate and methacrylate copolymers and graft copolymers of polyethyleneglycol, polyvinylcaprolactam, and polyvinylacetate;
neutral cellulosic polymers with at least one ester- and/or ether-linked substituent; preferably selected from hydroxypropyl methyl cellulose acetate, hydroxypropyl methyl cellulose, hydroxypropyl cellulose, methyl cellulose, ethyl cellulose, hydroxyethyl cellulose, hydroxyethyl methyl cellulose, hydroxyethyl cellulose acetate, and hydroxyethyl ethyl cellulose;
ionizable cellulosic polymers with at least one ester- and/or ether-linked substituent; preferably selected from hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate; and
amphiphilic cellulosic polymers obtainable by substituting the cellulose at any or all of the 3 hydroxyl substituents present on each saccharide repeat unit with at least one hydrophobic substituent; wherein said hydrophobic substituent is preferably selected from ether-linked alkyl groups and ester-linked alkyl groups, ether- and/or ester-linked aryl groups, and pheny late wherein besides the hydrophobic substituent(s) there may also be at least one hydrophilic substituents; wherein said hydrophilic substituent is preferably selected from ether- or ester-linked nonionizable groups, preferably hydroxy alkyl substituents, alkyl. ether groups, carboxylic acids, thiocarboxylic acids, substituted phenoxy groups, amines, phosphates or sulfonates.

In a particularly preferred embodiment, the one or more polymers comprise or essentially consist of a polyvinylpyrrolidone (PVP).

In another preferred embodiment, the one or more polymers comprise or essentially consist of a polyvinylpyrrolidone vinyl acetate copolymer (PVP/VA).

In another particularly preferred embodiment, the one or more polymers comprise or essentially consist of a hydroxypropylmethylcellulose (HPMC).

In still another particularly preferred embodiment, the one or more polymers comprise or essentially consist of a hydroxypropylmethylcellulose acetate succinate (HPMC-AS).

In yet another particularly preferred embodiment, the one or more polymers comprise or essentially consist of a polyethylene glycol, polyvinyl acetate and polyvinylcaprolactame-based graft copolymer (PVAc-PVCap-PEG).

In a preferred embodiment, the nanoparticles according to the invention comprise a polyoxypropylene-polyoxyethylene blockcopolymer, preferably poloxamer 407 as further described above; in combination with a polyoxyethylene ester of D-α-tocopheryl succinate; preferably D-α-tocopherol polyethylene glycol 1000 succinate.

In a preferred embodiment, the nanoparticles according to the invention comprise a polyoxy-propylene-polyoxyethylene blockcopolymer, preferably poloxamer 407 as further described above; in combination with a polyethylene glycol, polyvinyl acetate and polyvinylcaprolactame-based graft copolymer (PVAc-PVCap-PEG).

In a preferred embodiment, the nanoparticles according to the invention comprise a polyoxypropylene-polyoxyethylene blockcopolymer, preferably poloxamer 407 as further described above; in combination with polyvinylpyrrolidone (PVP).

In a preferred embodiment, the nanoparticles according to the invention comprise a polyoxypropylene-polyoxyethylene blockcopolymer, preferably poloxamer 407 as further described above 24; in combination with a hydroxypropylmethylcellulose (HPMC).

In a preferred embodiment, the nanoparticles according to the invention comprise a polyoxypropylene-polyoxyethylene block copolymer, preferably poloxamer 407 as further described above; in combination with a hydroxypropylmethylcellulose acetate succinate (HPMC-AS).

In a preferred embodiment, the nanoparticles according to the invention comprise an alkyl sulfate salt, preferably sodium dodecyl sulfate as further described above; in combination with a polyoxy ethylene ester of D-α-tocopheryl succinate; preferably D-α-tocopherol polyethylene glycol 1000 succinate.

In a preferred embodiment, the nanoparticles according to the invention comprise an alkyl sulfate salt, preferably sodium dodecyl sulfite as further described above; in combination with a polyethylene glycol, polyvinyl acetate and polyvinylcaprolactame-based graft copolymer (PVAc-PVCap-PEG).

In a preferred embodiment, the nanoparticles according to the invention comprise an alkyl sulfate salt, preferably sodium dodecyl sulfate as further described above; in combination with a polyvinylpyrrolidone (PVP).

In a preferred embodiment, the nanoparticles according to the invention comprise an alkyl sulfate salt, preferably sodium dodecyl sulfate as further described above; in combination with a hydroxypropylmethylcellulose (HPMC).

In a preferred embodiment, the nanoparticles according to the invention comprise an alkyl sulfate salt, preferably sodium dodecyl sulfate as further described above; in combination with a hydroxypropylmethylcellulose acetate succinate (HPMC-AS).

In a preferred embodiment, the nanoparticles according to the invention comprise a polyoxyethylene ester of D-α-tocopheryl succinate; preferably as further described above; in combination with a polyvinylpyrrolidone (PVP).

In a preferred embodiment, the nanoparticles according to the invention comprise a polyoxyethylene ester of D-α-tocopheryl succinate; preferably as further described above; in combination with a hydroxypropylmethylcellulose (HPMC).

In a preferred embodiment, the nanoparticles according to the invention comprise a polyoxyethylene ester of D-α-tocopheryl succinate; preferably as further described above; in combination with a hydroxypropylmethylcellulose acetate succinate (HPMC-AS).

In a preferred embodiment, the nanoparticles according to the invention comprise one or more phospholipids, which are preferably selected from phosphatidylcholines, phosphatidylglycerols, phosphatidylethanolamines, phosphatidylserines, phosphatidylinositol, and lecithins.

The term “phospholipids” typically refers to a class of lipids constituted of glycerol, a phosphate group, a neutral or zwitter-ionic moiety as the characterizing part (choline, serine, inositol etc); The glycerol moiety can be esterified with long chain fatty acids (C10-C22) which in turn can be saturated (e.g. myristic, palmitic and stearic acid), monounsaturated (e.g. oleic acid) or polyunsaturated (e.g. linoleic and arachidonic acid). For example, depending on the source, phosphatidylcholines include but are not limited to dilauryl-phosphatidylcholine, dimyristoyl-phosphatidylcholine, dipalmitoyl-phosphatidylcholine, palmitoyl-oleoyl-phosphatidylcholine, palmitoyl-linoleoyl-phosphatidylcholine, stearoyloleoyl-phosphatidylcholine, stearoyl-linoleoyl-phosphatidylcholine, and the like.

At least one of said phospholipids is preferably adsorbed on the surface of the Enzalutamide. For the purpose of the specification, the expression “adsorbed on the surface” typically means the adhesion of the phospholipid to the surface of the Enzalutamide. This process creates a film of the phospholipid on the surface. The adsorption of the phospholipid can be determined e.g. by differential scanning calorimetry (DSC), according to procedures known to the skilled person in the art. As for DSC analysis, the thermal trace of the preferably dried nanoparticles should not show the endothermal melting peak of Enzalutamide.

In another preferred embodiment, the nanoparticles according to the invention do not comprise any phospholipids.

In a preferred embodiment, the nanoparticles according to the invention comprise a cryoprotectant agent. Examples of cryoprotectant agents include but are not limited to mannitol, glycerol, propylene glycol, glycine, sucrose, lactose, trehalose, and mixtures thereof in any ratio by weight, preferably mannitol, trehalose and glycine, more preferably mannitol or trehalose or a mixture thereof in any weight ratio. Preferably, the cryoprotectant agent is a mixture of mannitol and trehalose in a ratio comprised between 6:4 and 4:6 by weight, more preferably in a 1:1 ratio by weight.

In another preferred embodiment, the nanoparticles according to the invention do not comprise a cryoprotectant agent.

Particularly preferred embodiments X¹ to X¹⁵ of the nanoparticles according to the invention are compiled in the following table:

| | X¹ | X² | X³ | X⁴ | X⁵ | X⁶ | X⁷ | X⁸ | X⁹ | X¹⁰ | X¹¹ | X¹² | X¹³ | X¹⁴ | X¹⁵ |
|---|---|---|---|---|---|---|---|---|---|---|---|---|---|---|---|
| Enzalutamide | + | + | + | + | + | + | + | + | + | + | + | + | + | + | + |
| sodium dodecyl | + | | | + | | | + | | | + | | | + | | |
| sulfate | | | | | | | | | | | | | | | |
| TPGS | | + | | | + | | | + | | | + | | | + | |
| poloxamer | | | + | | | + | | | + | | | + | | | + |
| PVAc-PVCap- | + | + | + | | | | | | | | | | | | |
| PEG | | | | | | | | | | | | | | | |
| PVP | | | | + | + | + | | | | | | | | | |
| PVP/VA | | | | | | | + | + | + | | | | | | |
| HPMC | | | | | | | | | | + | + | + | | | |
| HPMC-AS | | | | | | | | | | | | | + | + | + |

PVAc-PVCap-PEG: graft copolymer of polyethylene glycol, polyvinyl caprolactam, and polyvinyl acetate

Another aspect of the invention relates to a process for the preparation of nanoparticles according to the invention as described above involving precipitation of the nanoparticles from a liquid.

In a preferred embodiment, the process comprises the steps of
(a) providing a solution of Enzalutamide, optionally together with one or more pharmaceutical excipients, in a first liquid;
(b) providing a second liquid, optionally containing one or more pharmaceutical excipients in dissolved form; and
(c) contacting the first liquid and the second liquid thereby obtaining a third liquid comprising a mixture of the first liquid with the second liquid and precipitated nanoparticles.

The first liquid serves as a solvent for the Enzalutamide, whereas the second liquid typically serves as an antisolvent. The term “antisolvent” typically means a liquid having little or no solvation capacity for Enzalutamide. Once solvent and antisolvent are contacted, e.g. collided in form of jet streams, Enzalutamide is instantaneously precipitated thereby forming nanoparticles that are suspended in the combined first and second liquid.

Preferably, the solution provided in step (a) contains one or more surfactants as described above and/or one or more polymers as described above.

Preferably, the second liquid provided in step (b) contains one or more surfactants as described above and/or one or more polymers as described above.

Preferably, the amount of the Enzalutamide that is contained in the precipitated nanoparticles obtained in step (c) is at least 82 wt.-%, preferably at least 84 wt.-%, more preferably at least 86 wt.-%, in each case of the amount of Enzalutamide that was contained in the solution provided in step (a). Preferably, the amount of the Enzalutamide that is contained in the precipitated nanoparticles obtained in step (c) is at least 88 wt.-%, preferably at least 90 wt.-%, more preferably at least 92 wt.-%, in each case of the amount of Enzalutamide that was contained in the solution provided in step (a). Preferably, the amount of the Enzalutamide that is contained in the precipitated nanoparticles obtained in step (c) is at least 94 wt.-%, preferably at least 96 wt.-%, more preferably at least 98 wt.-%, in each case of the amount of Enzalutamide that was contained in the solution provided in step (a).

Preferably, the first liquid and the second liquid are mixed as jets that collide with each other at defined pressures and flow rates to effect instantaneous precipitation or co-precipitation during the course of which nanoparticles are formed.

In preferred embodiments of the process according to the invention, the particle size of the nanoparticles is controlled by
the temperature at which the first liquid and the second liquid are contacted,
the flow rate of the first liquid and the second liquid; and/or
pressure of a gas that is supplied to a reactor space of a microjet reactor wherein the first liquid and the second liquid are contacted; and/or
the concentration of the individual compounds in the solvent and antisolvent respectively,

The particle size can be adjusted by flow rates of the jet streams and the mixing ratio. At lower temperatures, solubility is reduced and the metastability zone is so narrow that supersaturation readily occurs if solvent is injected into the antisolvent. The nucleation process is a process whereby free energy is lost and heat is liberated: low temperatures thus promote a high nucleation rate. Lower temperatures can inhibit particle growth. The high nucleation rate and slow growth rate at low temperatures thus results in the formation of smaller particles. The finding that particle size and the degree of aggregation increase with increasing temperature may he explained by the fact that, as the temperature rises, the substance or additive is closer to its glass transition temperature. Particle size may also be controlled via solvent and antisolvent flow rates; small particles are obtained by selecting a high flow rate, large particles by selecting a low flow rate.

In preferred embodiments of the process according to the invention
the first liquid comprises a solvent selected from the group consisting of acetone, tetrahydrofuran, methanol, ethanol, isopropanol, and acetonitrile; preferably tetrahydrofuran or acetone; and/or
the second liquid comprises water; preferably the second liquid essentially consists of water and the optionally present pharmaceutical excipients.

In other preferred embodiments of the process according to the invention
the first liquid comprises glacial acetic acid; and/or
the second liquid comprises or essentially consists of an aqueous base; preferably the second liquid comprises or essentially consist of aqueous sodium hydroxide, aqueous potassium hydroxide or aqueous ammonia, and in each case the optionally present pharmaceutical excipients.

It has been found that the use of glacial acetic acid as solvent and an aqueous organic base as antisolvent can have advantages, as the solubilizing capacity of the glacial acetic acid for Enzalutamide can instantaneously be dropped upon contact with the aqueous base. In consequence, formation of amorphous Enzalutamide can be suppressed, i.e. the degree of crystallinity of Enzalutamide can be increased.

Preferably, the first liquid comprises a solvent for Enzalutamide and the second liquid comprises an antisolvent for Enzalutamide; wherein the contacting of the first liquid and the second liquid in step (c) generates the nanoparticies by controlled precipitation against the antisolvent using micro jet reactor technology.

In another preferred embodiment, the process comprises the steps of
(A) providing a solution of Enzalutamide in a first liquid, preferably not containing pharmaceutical excipients;
(B) providing a second liquid not containing pharmaceutical excipients;
(C) contacting the first liquid and the second liquid thereby obtaining a third liquid comprising a mixture of the first liquid with the second liquid and precipitated nanoparticles;
(D) providing a fourth liquid containing one or more pharmaceutical excipients in dissolved form; and
(E) contacting the third liquid and the fourth liquid thereby obtaining a fifth liquid comprising a mixture of the third liquid with the fourth liquid and precipitated coated nanoparticles which are coated with the one or more pharmaceutical excipients.

The first liquid serves as a solvent for the Enzalutamide, whereas the second liquid as well as the fourth liquid typically serve as an antisolvent.

Preferably, the solution provided in step (D) contains one or more surfactants as described above and/or one or more polymers as described above.

Preferably, the second liquid provided in step (B) essentially consists of an antisolvent and the fourth liquid provided in step (D) is a solution of the one or more pharmaceutical excipients in the same antisolvent; wherein the antisolvent is preferably water or an aqueous base.

Preferably, the precipitated nanoparticles obtained in step (C) essentially consist of Enzalutamide.

Preferably, the amount of the Enzalutamide that is contained in the precipitated coated nanoparticles obtained in step (E) is at least 82 wt.-%, preferably at least 84 wt.-%, more preferably at least 86 wt.-%, in each case of the amount of Enzalutamide that was contained in the solution provided in step (A). Preferably, the amount of the Enzalutamide that is contained in the precipitated coated nanoparticles obtained in step (E) is at least 88 wt.-%, preferably at least 90 wt.-%, more preferably at least 92 wt.-%, in each case of the amount of Enzalutamide that was contained in the solution provided in step (A). Preferably, the amount of the Enzalutamide that is contained in the precipitated coated nanoparticles obtained in step (E) is at least 94 wt.-%, preferably at least 96 wt.-%, more preferably at least 98 wt.-%, in each case of the amount of Enzalutamide that was contained in the solution provided in step (A).

Preferably, the first liquid and the second liquid, as well as the third liquid and the fourth liquid are mixed with One another as jets that collide with each other at defined pressures and flow rates, in step (C) to effect instantaneous precipitation or co-precipitation during the course of which nanoparticles are formed, and in step (E) to effect coating of the nanoparticles contained in the third liquid by instantaneous precipitation or co-precipitation of the one or more excipients contained in the fourth liquid. Preferably, the first liquid and the second liquid are mixed with one another as jets in a first microjet reactor, and the third liquid leaving said first microjet reactor is subsequently mixed with the fourth liquid in a second microjet reactor that is arranged downstream with respect to the first microjet reactor.

In preferred embodiments of the process according to the invention, the particle size of the nanoparticles is controlled by
the temperature at which the first liquid and the second liquid, and optionally the third liquid and the fourth liquid are contacted, and/or
the flow rate of the first liquid and the second liquid, and optionally of the third liquid and the fourth and/or
pressure of a gas that is supplied to a reactor space of a microjet reactor wherein the first liquid and the second liquid are contacted, and optionally pressure of a gas that is supplied to a reactor space of a microjet reactor wherein the third liquid and the fourth liquid are contacted; and/or
the concentration of the individual compounds in the solvent and antisolvent, respectively.

The particle size can be adjusted by flow rates of the jet streams and the mixing ratio. At lower temperatures, solubility is reduced and the metastability zone is so narrow that supersaturation readily occurs if solvent is injected into the antisolvent. The nucleation process is a process whereby free energy is lost and heat is liberated: low temperatures thus promote a high nucleation rate. Lower temperatures can inhibit particle growth. The high nucleation rate and slow growth rate at low temperatures thus results in the formation of smaller particles. The finding that particle size and the degree of aggregation increase with increasing temperature may be explained by the fact that, as the temperature rises, the substance or additive is closer to its glass transition temperature. Particle size may also be controlled via solvent and antisolvent flow rates; small particles arc obtained by selecting a high flow rate, large particles by selecting a low flow rate.

In preferred embodiments of the process according to the invention
the first liquid comprises a solvent selected from the group consisting of acetone, tetrahydrofuran, methanol, ethanol, isopropanol, and acetonitrile; preferably tetrahydrofuran or acetone; and/or
the second liquid comprises water; preferably the second liquid essentially consists of water and the optionally present pharmaceutical excipients; and/or
optionally, the fourth liquid comprises water; preferably the fourth liquid essentially consists of water and the optionally present pharmaceutical excipients.

In other preferred embodiments of the process according to the invention
the first liquid comprises glacial acetic acid; and/or
the second liquid comprises or essentially consists of an aqueous base; preferably the second liquid comprises or essentially consist of aqueous sodium hydroxide, aqueous potassium hydroxide or aqueous ammonia, and in each case the optionally present pharmaceutical excipients; and/or
optionally, the fourth liquid comprises or essentially consists of an aqueous base, preferably aqueous sodium hydroxide, aqueous potassium hydroxide or aqueous ammonia, and in each case the one or more pharmaceutical excipients.

It has been found that the use of glacial acetic acid as solvent and an aqueous organic base as antisolvent can have advantages, as the solubilizing capacity of the glacial acetic acid for Enzalutamide can instantaneously be dropped upon contact with the aqueous base. In consequence, formation of amorphous Enzalutamide can be suppressed, i.e. the degree of crystallinity of Enzalutamide can be increased.

Preferably, the first liquid comprises a solvent for Enzalutamide and the second liquid comprises an antisolvent for Enzalutamide; wherein the contacting of the first liquid and the second liquid in step (C) generates the nanoparticles by controlled precipitation against the antisolvent using micro jet reactor technology.

Preferably, in step (a) and (A) of the process according to the invention, Enzalutamide is employed in its non-salt form.

Preferably, the process according to the invention is performed by means of one or more microjet reactors. Preferably, each of said microjet reactors has at least two nozzles each of which has its own pump and feed line for injecting one liquid medium in each case into a reactor chamber enclosed in a reactor housing and on to a shared collision point, the reactor housing being provided with a first opening through which a gas can be introduced to promote the generation of the nanoparticle suspension and transportation of the suspension out of the reactor cell. further opening for removing the resulting products out of the reactor housing. The reactor includes all the geometries described in EP 1 165 224 111 and DE 10 2009 008 478 A1 which are incorporated herein by reference in their entirety.

The process makes use of controlled solvent/antisolvent precipitation in such a way that solvent (first liquid) and non-solvent (second liquid) streams with flow rates preferably exceeding 0.1 ml/min collide as impinging jets at a speed preferably greater than 1 m/s, more preferably greater than 50 m/s, and a Reynolds number of more than 100, preferably more than 500. Solvent and antisolvent are formed. in nozzles to jets which are preferably smaller than 1,000 μm, more preferably smaller than 500 μm and best of all smaller than 300 μm and have pressures generally of 1 bar, preferably in excess of 10 bar and even more preferably in excess of 50 bar, the pressure being controlled in this method by a pressure regulator. These two impinging jets collide in the microjet reactor in such a way as to effect precipitation at the point of collision of the jets, which, depending on the reactor geometry, form a double-disc-shaped structure there comprising fast-moving liquid jets. In the disc-edge area, very rapid mixing occurs at mixing speeds generally below 1 millisecond, frequently below 0.5 ns and mostly below 0.1 ms.

According to the invention, the microjet reactor can be used to produce nanoparticles by controlled precipitation, by co-precipitation and/or by self-organization processes. In the microjet reactor, the first liquid containing Enzalutamide in dissolved form and a the second liquid containing an antisolvent (nonsolvent) are mixed as jets that collide with each other in a microjet reactor at defined pressures and flow rates to effect very rapid precipitation or co-precipitation, during the course of which the nanoparticles are formed. As already mentioned above, the particle size can be controlled by the temperature at which the liquids collide, the flow rate of the liquids and/or the amount of gas. In general, smaller particle sizes are obtained at lower temperatures, at high liquid flow rates and/or in the complete absence of gas.

In a preferred embodiment, the process according to the invention comprises the following steps:
dissolving Enzalutamide in a water-miscible solvent, preferably under pressure, thereby obtaining a first liquid;
pumping the thus obtained solution (first liquid) through heated capillaries into a microjet reactor (e.g. precipitation reactor, free jet reactor, and the like),
colliding the liquid jet of the solution (first liquid) with a liquid jet formed by another nozzle of the microjet reactor (second liquid), the latter jet consisting of water or an aqueous solution of one or more excipients;
forming nanoparticulate nuclei by diffusion-controlled solvent/non-solvent precipitation at the collision point and the plate-like mixing zone of the liquid jets in a gaseous atmosphere.

The first liquid and the second liquid may be heated or cooled, namely by an external heating means or directly in the pump, in order to dissolve the Enzalutamide and/or the pharmaceutical excipient(s), to enable the formation of nanoparticles with the desired particle size and surface properties or to stabilize the resulting molecules.

As an alternative, another embodiment of the invention can use methods and an apparatus which allow self-organization processes in which one or more active target molecules react chemically with one or more suitable auxiliary agents that are soluble in the antisolvent, resulting in a product that is insoluble in the solvent/antisolvent mixture and thus permits the formation of microparticles or nanoparticles with sizes that vary according to parameters including, but not limited to, flow rate or concentration of the substances.

Methods for the manufacture of nanoparticles by colliding a solution of a drug in a solvent with a suitable antisolvent are known to the skilled person. In this regard it can be referred to e.g. EP-A 2. 550 092, EP-A 2 978 515 and EP-A 3 408 015 which are incorporated herein by reference in their entirety.

In a preferred embodiment the nanoparticles prepared in accordance with the invention remain in suspension of the mixture of the first liquid and the second liquid. This suspension can then advantageously used for manufacturing pharmaceutical dosage forms, e.g. by wet granulation wherein the solvent(s) contained in the first liquid and/or the solvents(s) contained in the second liquid serve as solvents in wet granulation, optionally together with additional solvents employed in the course of the wet granulation process.

Alternatively, the nanoparticles prepared in accordance with the invention in suspension of the mixture of the first liquid and the second liquid are spray dried and subsequently further processed.

Another aspect of the invention relates to nanoparticles that are obtainable by the process according to the invention as described above.

Another aspect of the invention relates to a pharmaceutical composition comprising the nanoparticles according to the invention as described above and one or more pharmaceutical excipients. Said one or more pharmaceutical excipients differ from the one or more surfactants and the one or more polymers as described above that are preferably contained in the nanoparticles according to the invention. Thus, said one or more pharmaceutical excipients of the pharmaceutical composition are present outside the nanoparticles.

It is contemplated that one or more pharmaceutical excipients that are contained in the nanoparticles according to the invention (e.g. surfactant and/or polymer) is also contained as pharmaceutical excipient in the pharmaceutical composition according to the invention, such that a first portion thereof is contained in the nanoparticles and the remainder thereof is contained outside the nanoparticles. In a preferred embodiment, however, the pharmaceutical excipients that are contained in the nanoparticles differ from the pharmaceutical excipients of the pharmaceutical composition, i.e. from those that are present outside the nanoparticles.

Preferably, the one or more pharmaceutical excipients form a matrix in which the nanoparticles are dispersed.

Preferably, the pharmaceutical excipients are selected from the group consisting of fillers, binders, disintegrants, surfactants, lubricants, glidants, retardant polymers and any combination thereof.

Examples of fillers (diluents) include but are not limited to starch, lactose, xylitol, sorbitol, confectioner's sugar, compressible sugar, dextrates, dextrin, dextrose, fructose, lactitol, mannitol, sucrose, talc, micro crystalline cellulose, calcium carbonate, calcium phosphate dibasic or tribasic, dicalcium phosphate dehydrate, calcium sulfate, and the like. Fillers typically represent from 2 wt.-% to 15 wt.-% of the pharmaceutical composition.

Examples of binders include but are not limited to starches such as potato starch, wheat starch, corn starch; microcrystalline cellulose; celluloses such as hydroxypropyl cellulose, hydroxyethyl cellulose, hydroxypropylmethylcellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, and the like. Binders typically represent from 0.2 wt.-% to 14 wt.-% of the composition.

Examples of disintegrants include, but are not limited to alginic acid, methacrylic acid DVB, cross-linked PVP, microcrystalline cellulose, sodium croscarmellose, crospovidone, polacrilin potassium, sodium starch glycolate, starch, including corn or maize starch, pregelatinized starch and the like. Disintegrant(s) typically represent from 2 wt.-% to 15 wt.-% of the pharmaceutical composition.

Examples of surfactants have already been described above in connection with the pharmaceutical excipients that are preferably contained in the nanoparticles. The same surfactants are principally also useful for the pharmaceutical composition according to the invention.

Examples of lubricants include, but are not limited to magnesium stearate, aluminum stearate, calcium stearate, zinc stearate, stearic acid, polyethylene glycol, glyceryl behenate, mineral oil, sodium stearyl fumarate, talc, hydrogenated vegetable oil and the like. Lubricants typically represent from 0.2 wt.-% to 5.0 wt.-% of the pharmaceutical composition.

Examples of glidants include but are not limited to silicon dioxide, colloidal anhydrous silica, magnesium trisilicate, tribasic calcium phosphate, calcium silicate, magnesium silicate, colloidal silicon dioxide, powdered cellulose, starch, talc, and the like. Glidants typically represent from 0.01 wt.-% to 0.3 wt.-% of the pharmaceutical composition.

Examples of retardant polymers include but are not limited to cellulose derivatives such as cellulose ethers or cellulose esters; guar and guar derivatives; pectin; carrageenan; xanthan gum; locust bean gum; agar; algin and its derivatives, gellan gum, acacia, starch and modified starches; and synthetic polymers; including but not limited to homo- and co-polymers of carboxyvinyl monomers, homo- and co-polymers of acrylates or methacrylate monomers, homo- and co-polymers of oxyethylene, or oxypropylene monomers; or any combination of the foregoing.

The weight content of the Enzalutamide in the pharmaceutical composition is not particularly limited. Preferably, the weight content of the Enzalutamide is at least 1.0 wt.-%, preferably at least 2.5 wt.-%, more preferably at least 5.0 wt.-%, in each case relative to the total weight of the pharmaceutical composition. Preferably, the weight content of the Enzalutamide is at least 10 wt.-%, preferably at least 15 wt.-%, more preferably at least 20 wt.-%, still more preferably at least 25 wt.-%, yet more preferably at least 30 wt.-%, even more preferably at least 35 wt.-%, still more preferably at least 40 wt.-%, most preferably at least 45 wt.-%, and in particular at least 50 wt.-%, in each case relative to the total weight of the pharmaceutical composition.

Another aspect of the invention relates to a pharmaceutical dosage form comprising the nanoparticles according to the invention as described above or the pharmaceutical composition according to the invention as described above.

Preferably, the pharmaceutical dosage form is selected from tablets, micro tablets, micro tablets, capsules, powders, granules, suspensions, emulsions.

In a particularly preferred embodiment, the pharmaceutical dosage form is a tablet, which is preferably granulated, more preferably wet-granulated. Preferably, the first and second liquid that are preferably employed in the preparation of the nanoparticles according to the invention as described above serve as granulation liquid for wet granulation.

Thus, preferably, wet granulation involves a liquid comprising water and a solvent selected from the group consisting of acetone, tetrahydrofuran, methanol, ethanol, isopropanol, and acetonitrile; preferably tetrahydrofuran or acetone.

In a preferred embodiment, the pharmaceutical dosage form according to the invention is film-coated tablet.

The total weight of the pharmaceutical dosage form according to the invention is not particularly limited. However, as far as oral dosage forms are concerned, the size should preferably not exceed a certain limit for ease of swallowing and patient compliance.

Preferably, the pharmaceutical dosage form has a total weight of not more than 1000 mg, preferably not more than 950 mg, more preferably not more than 900 mg, still more preferably not more than 850 mg, yet more preferably not more than 800 mg, even more preferably not more than 750 mg, most preferably not more than 700 mg, and in particular not more than 650 mg.

The dose of the Enzalutamide that is contained in the pharmaceutical dosage form according to the invention is not particularly limited and typically may depend upon the age and weight of the patient as well as on the severity of the disease or disorder or condition to be treated.

In preferred embodiments, the pharmaceutical dosage form according to the invention contains the Enzalutamide at a dose within the range of 30±15 mg, or 40±20 mg, or 60±30 mg, or 80±40 mg, or 120±60 mg, or 150±75 mg, or 160±80 mg, or 200±80 mg, or 240±120 mg, or 300±150 mg, or 360±180 mg, in each case expressed as weight equivalent of the non-salt form of Enzalutamide.

Preferably, the pharmaceutical dosage form according to the invention has a disintegration time in accordance with Ph. Eur. of not more than 8.0 minutes, preferably not more than 7.0 minutes, more preferably not more than 6.0 minutes, still more preferably not more than 5.0 minutes, yet more preferably not more than 4.0 minutes, even more preferably not more than 3.0 minutes, most preferably not more than 2.0 minutes, and in particular not more than 1.0 minute.

Preferably, the pharmaceutical dosage form according to the invention provides in accordance with Ph. Eur immediate release of the Enzalutamide, such that under in vitro conditions at 37° C., at pH 1.2 in 600 mL artificial gastric juice using a paddle apparatus at a rotational speed of 75 rpm has released after 30 minutes at least 80 wt.-%, preferably at least 85 wt.-%, more preferably at least 90 wt.-%, in each case of the Enzalutamide that was originally contained in the pharmaceutical dosage form.

Preferred immediate release profiles A1 to A40 of the pharmaceutical dosage form according to the invention under in vitro conditions at 37° C., at pH 1.2 in 600 mL artificial gastric juice using a paddle apparatus at a rotational speed of 75 rpm are compiled in the following table, wherein all percentages are based upon the weight of Enzalutamide that was originally contained in the pharmaceutical dosage form:

| | A1 | A2 | A3 | A4 | A5 | A6 | A7 | A8 | A9 | A10 |
|---|---|---|---|---|---|---|---|---|---|---|
| time | [%] | [%] | [%] | [%] | [%] | [%] | [%] | [%] | [%] | [%] |
| 15 min | ≥30 | ≥35 | ≥40 | ≥45 | ≥50 | ≥55 | ≥60 | ≥65 | ≥70 | ≥75 |
| 30 min | ≥80 | ≥80 | ≥80 | ≥80 | ≥80 | ≥>80 | ≥80 | ≥80 | ≥80 | ≥80 |
| | A11 | A12 | A13 | A14 | A15 | A16 | A17 | A18 | A19 | A20 |
| 15 min | ≥30 | ≥35 | ≥40 | ≥45 | ≥50 | ≥55 | ≥60 | ≥65 | ≥70 | ≥75 |
| 30 min | ≥85 | ≥85 | ≥85 | ≥85 | ≥85 | ≥85 | ≥85 | ≥85 | ≥85 | ≥85 |
| | A21 | A22 | A23 | A24 | A25 | A26 | A27 | A28 | A29 | A30 |
| 15 min | ≥30 | ≥35 | ≥40 | ≥45 | ≥50 | ≥55 | ≥60 | ≥65 | ≥70 | ≥75 |
| 30 min | ≥90 | ≥90 | ≥90 | ≥90 | ≥90 | ≥90 | ≥90 | ≥90 | ≥90 | ≥90 |
| | A31 | A32 | A33 | A34 | A35 | A36 | A37 | A38 | A39 | A40 |
| 15 min | ≥30 | ≥35 | ≥40 | ≥45 | ≥50 | ≥55 | ≥60 | ≥65 | ≥70 | ≥75 |
| 30 min | ≥95 | ≥95 | ≥95 | ≥95 | ≥95 | ≥95 | ≥95 | ≥95 | ≥95 | ≥95 |

Preferably, the pharmaceutical dosage form according to the invention provides an average oral bioavailability of Enzalutamide of at least 5%, preferably at least 10%, more preferably at least 15%, still more preferably at least 20%, yet more preferably at least 25%, even more preferably at least 30%, most preferably at least 35%, and in particular at least 40%. Preferably, the pharmaceutical dosage form according to the invention provides an average oral bioavailability of Enzalutamide of at least 50%, preferably at least 60%, more preferably at least 70%, still more preferably at least 80%, yet more preferably at least 85%, even more preferably at least 90%, most preferably at least 95%, and in particular at least 98%.

Preferably, the pharmaceutical dosage form according to the invention upon oral administration
at an administered dose of 30 mg provides a Cₘₐₓ of 0.4±0.1 μg/mL; and/or a tₘₐₓ within the range of 0.4 to 4 h; and/or an AUC_{∞} of 54±21 μg·h/mL; and/or
at an administered dose of 40 mg provides a Cₘₐₓ of 0.9±0.5 μg/mL; and/or a tₘₐₓ within the range of 0.4 to 4 h; and/or an AUC_{∞} of 65±30 μg·h/mL; and/or
at an administered dose of 60 mg provides a Cₘₐₓ of 1.7±0.5 μg/mL; and/or a tₘₐₓ within the range of 0.5 to 1 h; and/or an AUC_{∞} of 94±17 μg·h/mL; and/or
at an administered dose of 80 mg provides a Cₘₐₓ of 2.2±0.8 μg/mL; and/or a tₘₐₓ within the range of 0.5 to 2 h; and/or an AUC_{∞} of 120±40 μg·h/mL; and/or
at an administered dose of 150 mg provides a Cₘₐₓ of 3.4±0.8 μg/mL; and/or a tₘₐₓ within the range of 0.5 to 2 h; and/or an AUC_{∞} of 334±50 μg·h/mL; and/or
at an administered dose of 160 mg provides a Cₘₐₓ of 3.5±0.8 μg/mL; and/or a tₘₐₓ within the range of 0.5 to 2 h; and/or an AUC_{∞} of 400±50 μg·h/mL.

In another preferred embodiment of the invention, the pharmaceutical dosage form comprises the nanoparticles according to the invention provides controlled release of Enzalutamide, preferably prolonged release (sustained release, retarded release). Prolonged release is preferably achieved by matrix retardation, whereas other known measures to achieve retardation of release are also contemplated such as by means of suitable coatings optionally containing suitable pore formers and the like. Preferably, the pharmaceutical dosage form comprises a controlled release matrix material in which the nanoparticles according to the invention are embedded. Preferably, the controlled release matrix material comprises one or more retardant polymers.

In preferred embodiments, the controlled release matrix material comprises one or more polysaccharides independently of one another selected from cellulose derivatives such as cellulose ethers or cellulose esters; including but not limited to methylcellulose (MC), ethylcellulose (EC), carboxymethylcellulose (CMC), carboxymethylhydroxyethylcellulose (CMHEC), hydroxyethylcellulose (HEC), ethylhydroxyethylcellulose (EHEC), hydroxyethylmethylcellulose (HEMC), hydroxypropylcellulose (HPC), hydroxypropylmethylcellulose (HPMC), hydrophobically modified hydroxyethylcellulose (HMHEC), hydrophobically modified ethylhydroxyethylcellulose (HMEHEC), carboxymethyl hydrophobically modified hydroxyethylcellulose (CMHMHEC), and the like; guar and guar derivatives; pectin; carrageenan; xanthan gum; locust bean gum; agar; algin and its derivatives, gellan gum, acacia, starch and modified starches; or any combination of the foregoing.

In preferred embodiments, the controlled release matrix material comprises one or more synthetic polymers; including but not limited to homo- and co-polymers of carboxyvinyl monomers, homo- and co-polymers of acrylates or methacrylate monomers, homo- and co-polymers of oxyethylene, or oxypropylene monomers; or any combination of the foregoing.

The controlled release matrix material may contain additional pharmaceutical excipients such as fillers, binders, disintegrants, surfactants, lubricants, glidants, and any combination thereof, as defined above.

Preferred controlled release profiles B1 to B40 of the pharmaceutical dosage form according to the invention under in vitro conditions at 37° C., at pH 1.2 in 600 mL artificial gastric juice using a paddle apparatus at a rotational speed of 75 rpm are compiled in the following table, wherein all percentages are based upon the weight of Enzalutamide that was originally contained in the pharmaceutical dosage form:

| | B1 | B2 | B3 | B4 | B5 | B6 | B7 | B8 | B9 | B10 |
|---|---|---|---|---|---|---|---|---|---|---|
| time | [%] | [%] | [%] | [%] | [%] | [%] | [%] | [%] | [%] | [%] |
| 120 min | ≤10 | ≤15 | ≤20 | ≤25 | ≤30 | ≤35 | ≤40 | ≤45 | ≤50 | ≤55 |
| 240 min | 20 ± 15 | 20 ± 15 | 20 ± 15 | 40 ± 15 | 40 ± 15 | 20 ± 10 | 20 ± 10 | 20 ± 10 | 40 ± 10 | 40 ± 10 |
| 360 min | 40 ± 15 | 60 ± 15 | 80 ± 15 | 60 ± 15 | 80 ± 15 | 40 ± 10 | 60 ± 10 | 80 ± 10 | 60 ± 10 | 80 ± 10 |
| 720 min | ≥80 | ≥80 | ≥80 | ≥80 | ≥80 | ≥80 | ≥80 | ≥80 | ≥80 | ≥80 |
| | B11 | B12 | B13 | B14 | B15 | B16 | B17 | B18 | B19 | B20 |
| 120 min | ≤10 | ≤15 | ≤20 | ≤25 | ≤30 | ≤35 | ≤40 | ≤45 | ≤50 | ≤55 |
| 240 min | 20 ± 15 | 20 ± 15 | 20 ± 15 | 40 ± 15 | 40 ± 15 | 20 ± 10 | 20 ± 10 | 20 ± 10 | 40 ± 10 | 40 ± 10 |
| 360 min | 40 ± 15 | 60 ± 15 | 80 ± 15 | 60 ± 15 | 80 ± 15 | 40 ± 10 | 60 ± 10 | 80 ± 10 | 60 ± 10 | 80 ± 10 |
| 720 min | ≥90 | ≥90 | ≥90 | ≥90 | ≥90 | ≥90 | ≥90 | ≥90 | ≥90 | ≥90 |
| | B21 | B22 | B23 | B24 | B25 | B26 | B27 | B28 | B29 | B30 |
| 120 min | ≤5 | ≤10 | ≤15 | ≤20 | ≤25 | ≤30 | ≤35 | ≤40 | ≤45 | ≤50 |
| 240 min | 10 ± 5 | 20 ± 15 | 30 ± 15 | 10 ± 5 | 20 ± 15 | 10 ± 5 | 20 ± 10 | 30 ± 10 | 10 ± 5 | 20 ± 10 |
| 360 min | 30 ± 15 | 40 ± 15 | 50 ± 15 | 40 ± 15 | 50 ± 15 | 30 ± 10 | 40 ± 10 | 50 ± 10 | 40 ± 10 | 50 ± 10 |
| 720 min | 50 ± 15 | 60 ± 15 | 70 ± 15 | 60 ± 15 | 60 ± 15 | 50 ± 10 | 60 ± 10 | 70 ± 10 | 60 ± 10 | 60 ± 10 |
| 1440 min | ≥80 | ≥80 | ≥80 | ≥80 | ≥80 | ≥80 | ≥80 | ≥80 | ≥80 | ≥80 |
| | B31 | B32 | B33 | B34 | B35 | B36 | B37 | B38 | B39 | B40 |
| 120 min | ≤5 | ≤10 | ≤15 | ≤20 | ≤25 | ≤30 | ≤35 | ≤40 | ≤45 | ≤50 |
| 240 min | 10 ± 5 | 20 ± 15 | 30 ± 15 | 10 ± 5 | 20 ± 15 | 10 ± 5 | 20 ± 10 | 30 ± 10 | 10 ± 5 | 20 ± 10 |
| 360 min | 30 ± 15 | 40 ± 15 | 50 ± 15 | 40 ± 15 | 50 ± 15 | 30 ± 10 | 40 ± 10 | 50 ± 10 | 40 ± 10 | 50 ± 10 |
| 720 min | 50 ± 15 | 60 ± 15 | 70 ± 15 | 60 ± 15 | 60 ± 15 | 50 ± 10 | 60 ± 10 | 70 ± 10 | 60 ± 10 | 60 ± 10 |
| 1440 min | ≥90 | ≥90 | ≥90 | ≥90 | ≥90 | ≥90 | ≥90 | ≥90 | ≥90 | ≥90 |

Another aspect of the invention relates to a process for the preparation of the pharmaceutical dosage form according to the invention as described above comprising the steps of
(i) providing nanoparticles according to the invention as described above;
(ii) granulating, preferably wet-granulating the nanoparticles with one or more pharmaceutical excipients; and
(iii) compressing the granulate.

Preferably, the process for the preparation of the pharmaceutical dosage form comprises the process for the preparation of the nanoparticles according to the invention as described above.

In a preferred embodiment, step (ii) involves wet-granulating a third liquid comprising a mixture of a first liquid with a second liquid and precipitated nanoparticles as defined and preferably obtained in step (c) of the process for the preparation of the nanoparticles according to the invention as described above.

In another preferred embodiment, step (ii) involves wet-granulating a fifth liquid comprising a mixture of a third liquid with a fourth liquid and precipitated coated nanoparticles as defined preferably and obtained in step (E) of the process for the preparation of the nanoparticles according to the invention as described above.

Another aspect of the invention relates to the pharmaceutical dosage form according to the invention as described above for use in the treatment of a hyperproliferative disorder. Another aspect of the invention relates to a method of treating a hyperproliferative disorder comprising administering the pharmaceutical dosage form according to the invention as described above to a subject in need thereof. Another aspect of the invention relates to the use of Enzalutamide for the manufacture of a pharmaceutical dosage form according to the invention as described above for treating a hyperproliferative disorder.

Preferably, the hyperproliferative disorder is selected from the group consisting of benign prostatic hyperplasia, prostate cancer, breast cancer, and ovarian cancer. Preferably, the hyperproliferative disorder is prostate cancer selected from hormone-refractory prostate cancer and hormone-sensitive prostate cancer.

Preferably, the pharmaceutical dosage form according to the invention is administered orally.

Preferably, the pharmaceutical dosage form according to the invention is administered once daily or twice daily; preferably once daily; in each case optionally involving simultaneous administration of a plurality of pharmaceutical dosage forms. In this regard “simultaneous administration” means that more than one pharmaceutical dosage form is taken by a subject within a relatively short period of time, e.g. within 10 minutes, preferably within 5 minutes.

In a preferred embodiment, the pharmaceutical dosage form according to the invention is orally administered after a meal. In another preferred embodiment, the pharmaceutical dosage form according to the invention is orally administered before a meal.

The following examples further illustrate the invention but are not to be construed as limiting its scope:

### EXAMPLE 1

### Pluronic vs. SDS and Soluplus vs. PVP vs. TPGS vs. HPMC—Preliminary Precipitation Experiment

Eight formulations were prepared containing Enzalutamide in the non-salt form and various excipients. Precipitation experiments were performed. Acetone was used as solvent, water as non-solvent, and precipitation was effected under stirring. The particle size of the thus obtained precipitates was measured. The composition of the various formulations and the results of the particle size measurements are summarized in the table here below:

| | 1-1a | 1-1b | 1-2a | 1-2b | 1-3a | 1-3b | 1-4a | 1-4b |
|---|---|---|---|---|---|---|---|---|
| SDS | + | | + | | + | | + | |
| Pluronic ® F127 | | + | | + | | + | | + |
| Soluplus ® | + | + | | | | | | |
| PVP K30 | | | + | + | | | | |
| Kolliphor ® TPGS | | | | | + | + | | |
| HPMC | | | | | | | + | + |
| z-average particle | ~1600 | ~2000 | ~2200 | ~1100 | ~5500 | ~4500 | ~1900 | ~2600 |
| size [nm] | | | | | | | | |
| max [nm] | ~5000 | ~5000 | ~6200 | ~2800 | ~6500 | ~6200 | ~3800 | ~9000 |
| min [nm] | ~600 | ~120 | ~550 | ~800 | ~2100 | ~2300 | ~220 | ~1800 |

Due to the experimental design, particle size distributions were comparatively broad. The precipitate of formulations 1-1a, 1-1b, 1-2a, 1-2b, and 1-4a from acetone included at the lower end of the particle size distribution particles having an individual size of 800 nm or less (min [nm]) which was considered as the upper target limit. Based upon this preliminary test, formulations 1-1b (Pluronic F127/Soluplus), 1-2a (SDS/PVP K30) and 1-4a (SDS/HPMC) were considered to provide the best precipitate from acetone upon stirring.

### EXAMPLE 2

### Pluronic F127/Soluplus—Acetone vs. THF—Preliminary Precipitation Experiment

The system Pluronic F127/Soluplus was further investigated. Eight formulations were prepared containing different amounts of Enzalutamide in the non-salt form and different amounts of Pluronic F127. The concentration of Soluplus was kept constant (70 mg/ml). Precipitation experiments were performed from acetone and from tetrahydrofuran (THF) as solvent and water as non-solvent at a solvent to non-solvent ratio of ⅓. The z-average particle size of the thus obtained precipitates was measured.

It was further investigated whether the thus obtained particles disperse from suspension into fasted state simulating fluid (FaSSIF). For that purpose, 40 mg of particles were stirred in 900 ml of FaSSIF for 1 hour at 50 rpm and 37° C. The dispersion was then filtered through a 0.45 μm PTFE filter and the percentage of particles that were dispersed in FaSSIF was quantified.

The composition of the various formulations, the solvents, the results of the particle size measurements as well as the results of the dispersion experiments are summarized in the table here below:

| | 2-1 | 2-2 | 2-3 | 2-4 | 2-5 | 2-6 | 2-7 | 2-8 |
|---|---|---|---|---|---|---|---|---|
| Pluronic ® F127 [mg/ml] | 1 | 1 | 5 | 10 | 1 | 1 | 1 | 1 |
| Enzalutamide [mg/ml] | 300 | 300 | 300 | 300 | 300 | 200 | 100 | 50 |
| Soluplus ® [mg/ml] | 70 | 70 | 70 | 70 | 70 | 70 | 70 | 70 |
| solvent | acetone | THF | THF | THF | THF | THF | THF | THF |
| z-average particle size [nm] | ~120 | ~250 | ~100 | ~130 | ~250 | ~170 | ~150 | ~80 |
| dispersion in FaSSIF [%] | ~5 | ~15 | n.d. | ~25 | ~15 | ~90 | ~98 | ~92 |

Thus, precipitation from THF generally provided better results than precipitation from acetone. Further, formulations 2-6, 2-7 and 2-8 provided promising dispersion experiments in FaSSIF.

### EXAMPLE 3

### PVP K30/SDS/THF—Preliminary Precipitation Experiment

The system PVP K30/SDS (and PVP K30/Pluronic F127) was also further investigated. Five formulations were prepared containing different amounts of Enzalutamide in the non-salt form and different amounts of PVP K30. The concentration of SDS was kept constant. Precipitation experiments were performed from tetrahydrofuran (THF) as solvent and water as non-solvent at a solvent to nonsolvent ratio of ½. The z-average particle size of the thus obtained precipitates was measured.

It was further investigated in accordance with Example 2 whether the thus obtained particles disperse from suspension into fasted state simulating fluid (FaSSIF).

The composition of the various formulations, the solvents, the results of the particle size measurements as well as the results of the dispersion experiments are summarized in the table here below:

| | 3-1 | 3-2 | 3-3 | 3-4 | 3-5 |
|---|---|---|---|---|---|
| Pluronic ® F127 [mg/ml] | 5 | | | | |
| SDS [mg/ml] | | 10 | 10 | 10 | 10 |
| Enzalutamide [mg/ml] | 300 | 300 | 300 | 300 | 200 |
| PVP K30 [mg/ml] | 25 | 25 | 60 | 25 | 25 |
| solvent | THF | THF | THF | THF | THF |
| z-average particle size [nm] | ~320 | ~190 | ~310 | ~190 | ~140 |
| dispersion in FaSSIF [%] | n.d. | ~25 | n.d. | ~25 | ~45 |

### EXAMPLE 4

### HMPC/SDS/THF—Preliminary Precipitation Experiment

The system HPMC/SDS was also further investigated. Three formulations were prepared containing Enzalutamide in the non-salt form. The concentrations of SDS (60 mg/ml) and HPMC (25 mg/ml) were kept constant. Precipitation experiments were performed from acetone and tetrahydrofuran (THF) as solvent and water as non-solvent at different solvent to non-solvent ratios. The z-average particle size of the thus obtained precipitates was measured.

It was further investigated in accordance with Example 2 whether the thus obtained particles disperse from suspension into fasted state simulating fluid (FaSSIF).

The composition of the various formulations, the solvents, the results of the particle size measurements as well as the results of the dispersion experiments are summarized in the table here below:

| | 4-1 | 4-2 | 4-3 |
|---|---|---|---|
| HPMC [mg/ml] | 25 | 25 | 25 |
| SDS [mg/ml] | 60 | 60 | 60 |
| Enzalutamide [mg/ml] | 300 | 300 | 300 |
| solvent | acetone | THF | THF |
| ratio solvent/non-solvent | 1/3 | 1/3 | 1/2 |
| z-average particle size [nm] | ~280 | ~120 | ~80 |
| dispersion in FaSSIF [%] | ~10 | ~70 | ~98 |

Thus, Example 4-3 shows good particle sizes (z-average ˜80 nm) and disperses to a high degree (˜98%) from suspension into FaSSIF.

### EXAMPLE 5

### PVP K30/TPGS/THF—Preliminary Precipitation Experiment

The system PVP K30/TPGS was also investigated. Seven formulations were prepared containing Enzalutamide in the non-salt form. The concentrations of PVP K30 (60 mg/ml) and Enzalutamide (200 mg/ml) were kept constant. Precipitation experiments were performed from tetrahydrofuran (THF) as solvent and water as non-solvent at a solvent to non-solvent ratio of ⅓. The non-solvent contained the TPGS. The z-average particle size of the thus obtained precipitates was measured.

It was further investigated in accordance with Example 2 whether the thus obtained particles disperse from suspension into fasted state simulating fluid (FaSSIF).

The composition of the various formulations, the solvents, the results of the particle size measurements as well as the results of the dispersion experiments are summarized in the table here below:

| | 5-1 | 5-2 | 5-3 | 5-4 | 5-5 | 5-6 | 5-7 |
|---|---|---|---|---|---|---|---|
| TPGS [mg/ml] | 2.5 | 5 | 10 | 20 | 40 | 50 | 100 |
| Enzalutamide [mg/ml] | 200 | 200 | 200 | 200 | 200 | 200 | 200 |
| PVP K30 [mg/ml] | 25 | 25 | 25 | 25 | 25 | 25 | 25 |
| solvent | THF | THF | THF | THF | THF | THF | THF |
| z-average particle size [nm] | ~700 | ~450 | ~450 | ~280 | ~450 | ~690 | ~760 |
| dispersion in FaSSIF [%] | n.d. | n.d. | n.d. | n.d. | n.d. | ~15% | ~40% |

### EXAMPLE 6

### HPMC/TPGS/THF—Preliminary Precipitation Experiment

The system HPMC/TPGS was also investigated. Seven formulations were prepared containing Enzalutamide in the non-salt form. The concentrations of HPMC (60 mg/ml) and Enzalutamide (200 mg/ml) were kept constant. Precipitation experiments were performed from tetrahydrofuran (THF) as solvent and water as non-solvent at a solvent to non-solvent ratio of ⅓. The non-solvent contained the TPGS. The z-average particle size of the thus obtained precipitates was measured.

It was further investigated in accordance with Example 2 whether the thus obtained particles disperse from suspension into fasted state simulating fluid (FaSSIF).

The composition of the various formulations, the solvents, the results of the particle size measurements as well as the results of the dispersion experiments are summarized in the table here below:

| | 6-1 | 6-2 | 6-3 | 6-4 | 6-5 | 6-6 | 6-7 |
|---|---|---|---|---|---|---|---|
| TPGS [mg/ml] | 2.5 | 5 | 10 | 20 | 40 | 50 | 100 |
| Enzalutamide [mg/ml] | 200 | 200 | 200 | 200 | 200 | 200 | 200 |
| HPMC [mg/ml] | 25 | 25 | 25 | 25 | 25 | 25 | 25 |
| solvent | THF | THF | THF | THF | THF | THF | THF |
| z-average particle size [nm] | ~950 | ~800 | ~1550 | ~1500 | ~300 | ~320 | ~300 |
| dispersion in FaSSIF [%] | ~75% | n.d. | n.d. | n.d. | ~90 | ~93 | ~88 |

Thus, Examples 6-5 to 6-7 show good particle sizes and disperses to a high degree from suspension into FaSSIF.

### EXAMPLE 7

### HPMC-AS/Acetone—Preliminary Precipitation Experiment Coprecipitation

The system HPMC/TPGS was also investigated. Nine formulations were prepared containing Enzalutamide in the non-salt form. The concentrations of HPMC-AS (15 mg/ml) and Enzalutamide (either without or 5 mg/ml) were kept constant. Precipitation experiments were performed from acetone as solvent and water as non-solvent at a solvent to non-solvent ratio of ⅕. The water contained buffer at a concentration of 50 mM with various pH values. The z-average particle size of the thus obtained precipitates was measured.

It was further investigated in accordance with Example 2 whether the thus obtained particles disperse from suspension into fasted state simulating fluid (FaSSIF).

The composition of the various formulations, the solvents, the results of the particle size measurements as well as the results of the dispersion experiments are summarized in the table here below:

| | 7-1 | 7-2 | 7-3a | 7-3b | 7-4a | 7-4b | 7-5a | 7-5b | 7-6 |
|---|---|---|---|---|---|---|---|---|---|
| Enzalutamide [mg/ml] | w/o | w/o | w/o | 5 | w/o | 5 | w/o | 5 | 5 |
| HPMC-AS [mg/ml] | 15 | 15 | 15 | 15 | 15 | 15 | 15 | 15 | 15 |
| buffer | water | Ac | Ac | Ac | Ac | Ac | Ac | Ac | KH₂PO₄ |
| pH | | 4 | 5 | | 6 | | 7 | | 6.5 |
| z-average particle size [nm] | n.d. | n.d. | 209.3 | n.d. | 384.3 | 423.2 | 346.7 | 582.3 | 223.6 |
| dispersion in FaSSIF [%] | n.d. | n.d. | n.d. | n.d. | n.d. | 51 | n.d. | 51 | n.d. |

Ac = acetate buffer

### EXAMPLE 8

### Microjet Reactor

Based upon the above described screening by preliminary precipitation experiments, two representative systems C-1 (Soluplus®/Pluronic® F127) and C-2 (HPMC/SDS) were identified and the corresponding formulations were processed by nanojet technology. Nanoparticles were produced by means of a microjet reactor at 25° C. from Enzalutamide in the non-salt form dissolved in tetrahydrofuran as solvent and water as non-solvent.

The composition of the various formulations, the solvents, the results of the particle size measurements as well as the results of the dispersion experiments are summarized in the table here below:

| | Soluplus/Pluronic | | | HPMC/SDS | | |
|---|---|---|---|---|---|---|
| | C-1 | 8-1a | 8-1b | C-2 | 8-2a | 8-2b |
| Enzalutamide [mg/ml] | 200 | 200 | 200 | 300 | 300 | 300 |
| Soluplus ® | 70 | 70 | 70 | | | |
| HPMC | | | | 25 | 25 | 25 |
| Pluronic ® F127 | 1 | 1 | 1 | | | |
| SDS | | | | 60 | 60 | 60 |
| ratio THF/water | 1/3 | | | 1/2 | | |
| flow ratio solvent/non- | | 60/ | 60/ | | 60/ | 60/ |
| solvent ml/min/ml/min | | 180 | 180 | | 120 | 120 |
| pin hole [μm] | | 400 | 400 | | 400 | 400 |
| z-average [nm] | 170 | 117 | 115 | 84 | 294 | 203 |
| PDI | 0.15 | 0.19 | 0.17 | 0.25 | 0.11 | 0.06 |
| dispersion [%] | 91.8 | n.d. | n.d. | 97.3 | n.d. | n.d. |
| Enzalutamide [mg] | 40 | n.d. | n.d. | 40 | n.d. | n.d. |
| excipient [mg] | 42.6 | n.d. | n.d. | 22.6 | n.d. | n.d. |
| nanoparticles [mg] | 82.6 | n.d. | n.d. | 62.7 | n.d. | n.d. |
| remaining mass for | 217.4 | n.d. | n.d. | 237.3 | n.d. | n.d. |
| blend* [mg] | | | | | | |
| nanoparticles % of total | 28 | n.d. | n.d. | 21 | n.d. | n.d. |
| volume suspension | 0.8 | n.d. | n.d. | 0.4 | n.d. | n.d. |
| per tablet, ml | | | | | | |
| liquid/solid | 3.4 | n.d. | n.d. | 1.7 | n.d. | n.d. |

*target table weight 300 mg

Experimental results for the system Soluplus®0/Plutonic® F127 are further illustrated in FIGS. 1 to 3.

FIG. 1 shows the z-average particle size in dependence of the concentration of Enzalutamide (API) in suspension. The results of the preliminary precipitation experiments (beaker) are marked with symbol “ [symbol] ”, whereas the results of microjet reactor technology (MJR) are marked with symbols “▪”.

FIG. 2 shows the percentage of dispersion in FaSSIF in dependence of the concentration of Enzalutamide (API) in suspension. The results of the preliminary precipitation experiments (beaker) are marked with symbol “ [symbol] ”, whereas the results of microjet reactor technology (MJR) are marked with symbols “▪”.

As demonstrated, under the given experimental conditions, the particle size is reduced by flash precipitation (MJR) compared to preliminary precipitation experiments (beaker). Further, increasing concentration of Enzalutamide (API) results in an increase of particle size thereby decreasing dispersion in FaSSIF.

FIG. 3 shows the percentage of dispersion in FaSSIF in dependence of the z-average particle size. The results of the preliminary precipitation experiments (beaker) are marked with symbol “ [symbol] ”, whereas the results of microjet reactor technology (MJR) are marked with symbols “▪”.

As demonstrated, small particles disperse better than bigger particles.

### EXAMPLE 9

### Microjet Reactor

Nanoparticles were produced from Enzalutamide in the non-salt form dissolved in tetrahydrofuran and from the pharmaceutical excipients Soluplus® and Pluronic® F127 dissolved in water. Thus, tetrahydrofuran was used as the solvent for the first liquid, whereas water is used as antisolvent for the second liquid containing Soluplus® and Pluronic® F127. A temperature of 25° C. was set for the first liquid, the second liquid and the microjet reactor (pin hole 400 μm, no pressure, flow rate 200 ml/min).

Particles with different particle sizes are produced. The results are compiled in the table here below:

| | 9-1 | 9-2 | 9-3 | 9-4 | 9-5 |
|---|---|---|---|---|---|
| Enzalutamide in THF [mg/ml] | 253 | 212 | 304 | 400 | 300 |
| Soluplus ® in THF [mg/ml] | — | — | — | — | 333.3 |
| Soluplus ® in water [mg/ml] | 157 | 188 | 200 | 200 | 200 |
| Pluronic ® F127 in water [mg/ml] | 1 | 1.3 | 1 | 1 | 2 |
| ratio THF/water | 4 | 4 | 4 | 4 | |
| z-average particle size [nm] | 108 (98.3) | 72.6 (77.8) | 89 (85.9) | 97 (112.5) | n.d. |
| dispersion [%] | 99 (99.6) | 99.1 (98.5) | 100 (98.9) | 90 (91.8) | n.d. |

Thus, Examples 9-1 to 9-4 show good particle sizes and disperse to a high degree from suspension into FaSSIF.

1. Nanoparticles comprising Enzalutamide.

2. (canceled)

3. The nanoparticles according to claim 1, wherein the Enzalutamide has a degree of crystallinity of at least 60%.

4. (canceled)

5. The nanoparticles according to claim 1, wherein Enzalutamide is the sole pharmacologically active ingredient that is contained in the nanoparticles.

6. The nanoparticles according to claim 1, which have a z-average particle size Dz determined in accordance with ISO 22412:2008 Particle Size Analysis—Dynamic Light Scattering of not more than 1000 nm.

7.-13. (canceled)

14. The nanoparticles according to claim 1, which further comprise one or more surfactants.

15.-21. (canceled)

22. The nanoparticles according to claim 14, wherein the one or more surfactants comprise nonionic surfactants selected from the group consisting of
straight or branched chain fatty alcohols;
sterols;
lanolin alcohols;
partial fatty acid esters of multivalent alcohols;
partial fatty acid esters of sorbitan;
partial fatty acid esters of polyoxyethylene sorbitan;
polyoxyethyleneglycerole fatty acid esters;
polyoxyethylene fatty acid esters;
fatty alcohol ethers of polyoxyethylene;
reaction products of a natural or hydrogenated castor oil and ethylene oxide; and
polyoxypropylene-polyoxyethylene blockcopolymers (poloxamers); 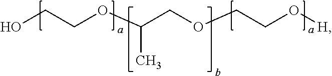
polyglycolyzed glycerides;
fatty acid esters of sucrose;
fatty acid esters of polyglycerol; and
polyoxyethylene esters of D-α-tocopheryl succinate.

23.-25. (canceled)

26. The nanoparticles according to claim 14, wherein the one or more surfactants comprise anionic surfactants selected from the group consisting of
alkyl sulfate salts;
fatty acid salts;
salts of cholic acid.

27.-28. (canceled)

29. The nanoparticles according to claim 1, which further comprise one or more polymers.

30.-35. (canceled)

36. The nanoparticles according to claim 29, wherein the one or more polymers comprise or essentially consist of a polymer selected from the group consisting of
neutral non-cellulosic polymers;
ionizable non-cellulosic polymers;
amphiphilic non-cellulosic polymers;
neutral cellulosic polymers with at least one ester- and/or ether-linked substituent;
ionizable cellulosic polymers with at least one ester- and/or ether-linked substituent; and
amphiphilic cellulosic polymers obtained by substituting the cellulose at any or all of the 3 hydroxyl substituents present on each saccharide repeat unit with at least one hydrophobic substituent.

37.-53. (canceled)

54. A process for the preparation of nanoparticles according to claim 1 comprising precipitation of the nanoparticles from a liquid.

55. The process according to claim 54 comprising the steps of
(i) (a) providing a solution of Enzalutamide, optionally together with one or more pharmaceutical excipients, in a first liquid;
(b) providing a second liquid, optionally containing one or more pharmaceutical excipients in dissolved form; and
(c) contacting the first liquid and the second liquid thereby obtaining a third liquid comprising a mixture of the first liquid with the second liquid and precipitated nanoparticles; or
(ii) (A) providing a solution of Enzalutamide in a first liquid, optionally not containing pharmaceutical excipients;
(B) providing a second liquid not containing pharmaceutical excipients;
(C) contacting the first liquid and the second liquid thereby obtaining a third liquid comprising a mixture of the first liquid with the second liquid and precipitated nanoparticles;
(D) providing a fourth liquid containing one or more pharmaceutical excipients in dissolved form; and
(E) contacting the third liquid and the fourth liquid thereby obtaining a fifth liquid comprising a mixture of the third liquid with the fourth liquid and precipitated coated nanoparticles which are coated with the one or more pharmaceutical excipients.

56.-66. (canceled)

67. Nanoparticles obtained by the process according to claim 54.

68. A pharmaceutical composition comprising nanoparticles according to claim 1 and one or more pharmaceutical excipients.

69.-72. (canceled)

73. A pharmaceutical dosage form comprising the nanoparticles according to claim 1.

74.-81. (canceled)

82. The pharmaceutical dosage form according to claim 73, which is a tablet and provides in accordance with Ph. Eur. immediate release of the Enzalutamide, such that under in vitro conditions at 37° C., at pH 1.2 in 600 mL artificial gastric juice using a paddle apparatus at a rotational speed of 75 rpm has released after 30 minutes at least 80 wt.-% of the Enzalutamide that was originally contained in the pharmaceutical dosage form.

83. (canceled)

84. The pharmaceutical dosage form according to claim 73, which upon oral administration
at an administered dose of 30 mg provides a Cₘₐₓ of 0.4±0.1 μg/mL; and/or a tₘₐₓ within the range of 0.4 to 4 h; and/or an AUG_{∞} of 54±21 μg·h/mL; and/or
at an administered dose of 40 mg provides a Cₘₐₓ of 0.9±0.5 μg/mL; and/or a tₘₐₓ within the range of 0.4 to 4 h; and/or an AUG_{∞} of 65±30 μg·h/mL; and/or
at an administered dose of 60 mg provides a Cₘₐₓ of 1.7±0.5 μg/mL; and/or a tₘₐₓ within the range of 0.5 to 1 h; and/or an AUG_{∞} of 94±17 μg·h/mL; and/or
at an administered dose of 80 mg provides a Cₘₐₓ of 2.2±0.8 μg/mL; and/or a tₘₐₓ within the range of 0.5 to 2 h; and/or an AUG_{∞} of 120±40 μg·h/mL; and/or
at an administered dose of 150 mg provides a Cₘₐₓ of 3.4±0.8 μg/mL; and/or a tₘₐₓ within the range of 0.5 to 2 h; and/or an AUG_{∞} of 334±50 μg·h/mL; and/or
at an administered dose of 160 mg provides a Cₘₐₓ of 3.5±0.8 μg/mL; and/or a tₘₐₓ within the range of 0.5 to 2 h; and/or an AUC_{∞} of 400±50 μg·h/mL.

85. A process for the preparation of the pharmaceutical dosage form according to claim 73 comprising the steps of (i) providing said nanoparticles; (ii) granulating, the nanoparticles with one or more pharmaceutical excipients to yield a granulate; and (iii) compressing the granulate.

86.-93. (canceled)

94. The nanoparticles according to claim 1, wherein the Enzalutamide contained there is not conjugated to an antigen nor are the nanoparticles encapsulated in or coated with an antigen.

95. A method of treating a hyperproliferative disorder in a patient in need of such treatment, said method comprising administering to said patient an effective amount therefor of the nanoparticles according to claim 1.
